(19) United States
(12) Patent Application Publication
(10) Pub. No.: US 2023/0340569 A1
(43) Pub. Date: Oct. 26, 2023

(54) METHODS FOR DETECTING PRIMARY IMMUNODEFICIENCY

(71) Applicant: Immunosis Pty Ltd, Princes Hill, Victoria (AU)

(72) Inventors: Simon Green, Melbourne (AU); Benjamin Cocks, Melbourne (AU)

(21) Appl. No.: 17/797,503

(22) PCT Filed: Feb. 5, 2021
(86) PCT No.: PCT/AU2021/050095

§ 371 Date: Aug. 4, 2022

(30) Foreign Application Priority Data
Feb. 7, 2020 (AU) 2020900337

(51) Int. Cl. C12Q 1/6809; G16B 25/10; G16H 50/30

(52) U.S. Cl. CPC C12Q 1/6809; G16B 25/10; G16H 50/30; G01N 2800/50; G01N 2800/24; C12Q 2600/118

(57) ABSTRACT

The invention relates to a method for determining whether a subject has or is susceptible to developing a primary immunodeficiency (PID), the method comprising using a linear mixed model to fit a transcriptome profile of the subject to a PID prediction equation developed by fitting into a linear mixed model a transcriptomic relationship matrix generated from a reference set of transcriptome profiles of reference subjects with and without PID, wherein the prediction equation's result indicates whether the subject has or is susceptible to PID. The invention relates to a method for developing a primary immunodeficiency (PID) prediction equation for determining whether a subject has or is susceptible to developing a PID, the method comprising fitting into a linear mixed model a transcriptomic relationship matrix generated from a reference set of transcriptome profiles of reference subjects with and without PID to develop the PID prediction equation.

### CROSS-REFERENCE TO EARLIER APPLICATION

This application claims priority from Australian patent application no. 2020900337 the entire content of which is incorporated by reference in its entirety.

### FIELD OF INVENTION

The present invention relates to methods for determining whether a subject has or is susceptible to developing a primary immunodeficiency (PID).

### BACKGROUND OF THE INVENTION

Primary immunodeficiencies (PID) are a group of diseases caused by congenital defects in the immune system, with as many as 200 different causative mutations known. PID is characterized by severe recurrent infections that can be life-threatening. Effective treatments, including hematopoietic stem cell transplantation, gene therapy, enzyme replacement therapy and intravenous immunoglobulins, are available for PID. Early diagnosis is critical for reducing disease-associated morbidity, treatment costs, and for improving patient outcomes. While the detailed clinical phenotype and molecular basis of an increasing number of immunological defects in PID have been determined, there still exists a need for a timely and accurate diagnosis in clinical practice.

Owing to the variety of clinical symptoms of PID and the complexity of current diagnostic procedures it takes an average of 5 years from symptom onset to diagnosis. The current diagnostic procedures involve a myriad of specialized, costly and laborious functional tests including lymphocyte proliferation and cytotoxicity assays, flow cytometry, measurement of serum immunoglobulin levels, complete blood cell counts, neutrophil function tests, and complement assays.

A number of DNA sequencing approaches have been explored for assisting diagnosing PID. Targeted Sanger or other gene exon sequencing or genotyping has been used to establish a PID classification and to devise an optimal treatment strategy. The selection of candidate genes to test is often guided by each patient's individual clinical and immunological characteristics. However, determining which genes (or specific mutations) to assess is not always clear, although largely a monogenic disease, over 200 different causative mutations have been described and there are likely several hundred more. Furthermore, mutations in different genes can manifest as similar phenotypes (locus heterogeneity), while mutations in different parts of the same gene can manifest as distinct phenotypes (allelic heterogeneity).

Next-generation sequencing (NGS), including whole genome sequencing (WGS) or whole exome sequencing (WES), has made it possible to simultaneously amplify and sequence millions of DNA fragments from a single subject within a few days. However, identification of causative mutations can be challenging because there are many nucleotide variants to measure and new variants that are detected by NGS are difficult to interpret since they often relate to poorly characterized genes or have unpredictable biological consequences on protein function.

A recognized limitation of DNA sequencing is that it does not provide functional information on the performance of the immune system, key information that is also required for PID diagnosis.

Gene expression analysis can provide insights into the functional consequence of PID mutations [1]. Salem et al 2014 reported that RNA sequencing of blood cells derived from a patient with the PID mutation IRF8^{K108E} revealed a reduced expression of IRF8-regulated target genes as well as a paucity of cell-type specific transcripts, indicative of cytopenias [1]. While useful as an investigatory tool, gene expression analysis alone is not used or thought of as a direct diagnosis approach for PID, with current approaches for diagnosis relying on cell-based functional information on the composition and performance of the immune system, combined with knowledge of the causative mutation if able to be determined. Functional insight gained from gene expression analysis may allow the identification of sets of genes whose expression is indicative of, and can discriminate, PID from immune competent individuals including individuals with other disorders of the immune system.

Importantly, a comprehensive analysis of gene expression levels in patients as a complex phenotype analysis has not been used or thought of as a direct diagnosis approach for PID. RNA sequencing has the advantage of being able to provide a measure of immune cell composition and activity with potential for diagnosis.

As noted above, a defining characteristic of PID is recurrent infection, owing to the inability of the immune system to manage microbial colonisation and invasion. While identification of specific pathogens may be useful and inform treatment in some cases, monitoring commensal microbial community composition may also provide useful information for managing PID. Microbial community is increasingly being shown to have a functional interaction with the immune system [2], including in the skin of PID patients [3], which appear to exhibit some fundamental differences.

There exists a need for an efficient and accurate diagnostic method for PID, which can be deployed at a reduced cost. This will have an impact on treatment decisions to improve survival and quality of life for patients, and a major public health impact by increasing rates and timeliness of diagnosis, thereby significantly reducing the cost of care for patients, and reduced demand on expensive pathology services.

Reference to any prior art in the specification is not an acknowledgement or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The inventors provide a method of determining whether a subject has or is susceptible to developing PID. The method comprises RNA analyses (RNAseq) of gene expression, i.e. the transcriptome, and optionally gene sequence mutations, and further comprises using a linear mixed model with RNA expression levels as input (not sequence or SNPs) to detect lack of function in the immune system reflected in the transcriptome, and optionally detection of specific PID sequence mutations. In addition, the inventors provide a method of using metagenome profiling as a measure of commensal microbial community structure in combination with RNA-sequencing mixed model analysis to determine whether a subject has or is susceptible to developing PID.

Accordingly, in one aspect the present invention provides a method for determining whether a subject has or is susceptible to developing a primary immunodeficiency (PID), the method comprising:
using a linear mixed model to fit a transcriptome profile of the subject to a PID prediction equation developed by fitting into a linear mixed model a transcriptomic relationship matrix generated from a reference set of transcriptome profiles of reference subjects with and without PID,
   wherein the prediction equation's result indicates whether the subject has or is susceptible to PID.

In another aspect the present invention provides a method for determining whether a subject has or is susceptible to developing a primary immunodeficiency (PID), the method comprising:
generating a transcriptome profile from the sample; and
using a linear mixed model to fit the transcriptome profile of the subject to a PID prediction equation developed by fitting into a linear mixed model a transcriptomic relationship matrix generated from a reference set of transcriptome profiles of reference subjects with and without PID,
   wherein the prediction equation's result indicates whether the subject has or is susceptible to PID.

In further aspect the present invention provides a method for determining whether a subject has or is susceptible to developing a primary immunodeficiency (PID), the method comprising:
obtaining a sample from a subject;
generating a transcriptome profile from the sample; and
using a linear mixed model to fit a transcriptome profile of the subject to a PID prediction equation developed by fitting into a linear mixed model a transcriptomic relationship matrix generated from a reference set of transcriptome profiles of reference subjects with and without PID,
   wherein the prediction equation's result indicates whether the subject has or is susceptible to PID.

In one aspect, the present invention provides a method for developing a primary immunodeficiency (PID) prediction equation for determining whether a subject has or is susceptible to developing a PID, the method comprising:
fitting into a linear mixed model a transcriptomic relationship matrix generated from a reference set of transcriptome profiles of reference subjects with and without PID to develop the PID prediction equation.

In another aspect, the present invention provides a method for developing a primary immunodeficiency (PID) prediction equation for determining whether a subject has or is susceptible to developing a PID, the method comprising:
generating a reference transcriptome profile from reference subjects;
generating a reference set of transcriptome profiles; and
fitting into a linear mixed model a transcriptomic relationship matrix generated from the reference set of transcriptome profiles of reference subjects with and without PID to develop the PID prediction equation.

In another aspect, the present invention provides a method for developing a primary immunodeficiency (PID) prediction equation for determining whether a subject has or is susceptible to developing a PID, the method comprising:
obtaining sample(s) from one or more subjects with and without PID;
generating a reference transcriptome profile from each subject;
generating a reference set of transcriptome profiles; and
fitting into a linear mixed model a transcriptomic relationship matrix generated from the reference set of transcriptome profiles of reference subjects with and without PID to develop the PID prediction equation.

In any embodiment of the above methods, the method further comprises measuring or determining the transcriptome profile of the subject for whom the determination of PID or susceptibility to PID is to be made.

In any embodiment, the reference set of transcriptome profiles and/or transcriptome profile of the subject for whom the determination of PID or susceptibility to PID is to be made includes at least 50, at least 100, at least 150, at least 200, at least 250, at least 300, at least 350, at least 400, at least 450, or all 500 of the genes listed in Table 1, Table 2, or Tables 1 and 2.

In a preferred embodiment of the invention, the linear mixed model is best linear unbiased prediction (BLUP), BayesR, or machine learning approaches. In a further embodiment of the invention, the machine learning approaches one of elastic net, ridge regression, lasso regression, random forest, gradient boosting machines, support vector machines, multilayer perceptrons (MLP) or convolutional neural networks (CNN).

In an embodiment of the invention, the PID prediction equation provides an absolute predictive score. In one embodiment, an absolute predictive score of greater than 0.2, greater than 0.4, greater than 0.6, or about 0.2, about 0.4 or about 0.6.

In an embodiment of the invention, the PID prediction equation provides a relative predictive score, wherein the relative score is calculated by subtracting the healthy control score (determined from a population of known healthy control subjects) from the patient score (determined from the sample of the subject to be diagnosed). In one embodiment, a relative predictive score is greater than 0, greater than 0.1 and greater than 0.2, or about 0, about 0.1 or about 0.2.

In an embodiment of the invention, where a known PID gene mutation is detected, an absolute predictive score of close to 1.0, preferably 1.0, can be designated.

In any embodiment of the invention, the PID prediction equation further provides a read-out of the PID gene mutation.

In any embodiment of the above methods, the reference set further comprises a RNA sequence mutation profile.

In any embodiment of the above methods, the method further comprises measuring or determining a RNA sequence mutation profile of the subject for whom the determination of PID or susceptibility to PID is to be made.

In any embodiment of the above methods, transcriptome profile is used to provide further information in relation to the defective pathway within the subject with PID. For example, a report may be generated that states the patient has a deficiency in the Fc receptor signalling pathway, the complement pathway or the Interferon signalling pathway. This provides information to the clinician that may assist with prescribing treatment options.

In preferred embodiments of the invention, the mutation profile comprises:
a) a RNA sequence of a PID gene comprising a known mutation associated with, involved in, or causative of, PID;
b) a new mutation, optionally a frameshift mutation or a missense amino acid changing mutation, or nonsense stop codon, that affects structure or function of a protein encoded by a known gene mutation associated with, involved in, or causative of, PID;
c) a dominant mutation in one allele that is associated with, involved in, or causative of, PID;
d) two different mutations in the same gene, but on two different alleles that are associated with, involved in, or causative of, PID;
e) a known mutation in RNA that is inferred or imputed by linkage to a co-occurring marker for a mutation associated with, involved in, or causative of, PID;
f) absence of expression of a gene normally expressed in non-PID subjects indicating a regulatory defect or destabilising mutation;
g) a defective exon structure indicating a splicing defect;
h) one or more, optionally one to three, additional mutations associated with, involved in, or causative of, PID; or
i) a sequence of more than one other gene, or an imputed sequence of more than one other gene, that associated with, involved in, or causative of, PID severity.

In any embodiment of the above methods, the reference set further comprises a DNA sequence mutation profile.

In any embodiment of the above methods, the method further comprises measuring or determining the DNA sequence mutation profile of the subject for whom the determination of PID or susceptibility to PID is to be made. Preferably, the linear mixed model is used to fit the transcriptome profile and the DNA sequence mutation profile of the subject to the PID prediction equation.

In any embodiment of the above methods, the reference set further comprises a metagenome profile and the linear mixed model is used to fit the transcriptome profile and a metagenome profile of the subject to the PID prediction equation.

In a preferred embodiment of the above methods, the metagenome profile is obtained from a mouth swab, nose swab, throat swab, saliva, faeces, or skin.

In a further preferred embodiment, the subject is human.

In a further aspect of the present invention a method determining whether a subject has or is susceptible to developing a primary immunodeficiency (PID), the method comprising using a linear mixed model to fit a metagenomics profile of the subject to a PID prediction equation developed by fitting into a linear mixed model a metagenomic relationship matrix generated from a reference set of metagenomic profiles of reference subjects with and without PID, wherein the prediction equation's result indicates whether the subject has or is susceptible to PID.

It will be understood that the transcriptome profile or sequence mutation profile is obtained from sputum, blood, amniotic fluid, plasma, semen, bone marrow, tissue, urine, peritoneal fluid, or pleural fluid, optionally obtained by fine needle biopsy.

It will be further understood that the transcriptome profile or sequence (DNA and/or RNA) mutation profile is generated in vitro or ex vivo.

It will be further understood that the transcriptome profile or sequence (DNA and/or RNA) mutation profile is generated in vitro, ex vivo, or in-silico.

In some embodiments of the above methods, the method is not practised on a human or animal body.

In some embodiments of the above methods, the method excludes any set of direct data acquisition practised on the human or animal body.

In a preferred embodiment of the above methods, the blood comprises peripheral blood mononuclear cells.

In any aspect or embodiment, the transcriptome, sequence (DNA and/or RNA) mutation profile and metagenome profile is determine from a sample previously obtained from the subject.

In another aspect, the present invention provides a method for determining whether a subject has or is susceptible to developing a primary immunodeficiency (PID), the method comprising using a linear mixed model to fit a transcriptome profile of the subject to a PID prediction equation developed by fitting into a linear mixed model a transcriptomic relationship matrix generated from a reference set of transcriptome profiles of reference subjects with and without PID, wherein the prediction equation's result indicates whether the subject has or is susceptible to PID.

In another aspect, the present invention provides a method of treating primary immunodeficiency (PID) in a subject who has or is susceptible to developing a primary immunodeficiency (PID), the method comprising:
determining whether the subject has or is susceptible PID by performing or having performed a method as described herein; and
wherein if the subject has or is susceptible PID then administering to the subject therapy specific to the PID.

In another aspect, the present invention provides a method of treating primary immunodeficiency (PID) in a subject who has or is susceptible to developing a primary immunodeficiency (PID), the method comprising:
determining whether the subject has PID by using a linear mixed model to fit a transcriptome profile of the subject to a PID prediction equation developed by fitting into a linear mixed model a transcriptomic relationship matrix generated from a reference set of transcriptome profiles of reference subjects with and without PID, wherein the prediction equation's result indicates whether the subject has or is susceptible to PID,
wherein if the subject has or is susceptible to developing a primary immunodeficiency (PID) then administering to the subject therapy specific to the PID.

In another aspect, the present invention provides a use of the therapy specific to primary immunodeficiency (PID) in the manufacture of a medicament for treating primary immunodeficiency (PID) in a subject who has or is susceptible to developing a primary immunodeficiency (PID), wherein the subject is diagnosed by the method as described herein.

In another aspect, the present invention provides a method of determining the efficacy of PID therapy in a subject, the method comprising:
providing a first sample obtained from a subject before receiving PID therapy;
providing a second sample obtained from a subject during, or after, receiving PID therapy;
using a linear mixed model to fit a transcriptome profile of the subject's first and second samples to a PID prediction equation developed by fitting into a linear mixed model a transcriptomic relationship matrix generated from a reference set of transcriptome profiles of reference subjects with and without PID, wherein the prediction equation's result indicates whether the subject has or is susceptible to PID
wherein the change in transcriptome profile from the first and second samples indicates efficacy of the PID therapy in the subject.

In one embodiment of the above methods, the PID therapy is intravenous immunoglobulin (IVIG) administration. In a further embodiment, the intravenous immunoglobulin (IVIG) is administered at a dose of between 200-800 mg/kg. In a further embodiment, the dose of intravenous immunoglobulin (IVIG) is administered every 3-4 weeks.

In another embodiment of the above methods, the PID therapy is subcutaneous immunoglobulin (SCIG) administration. In a further embodiment, the subcutaneous (SCIG) is administered either daily, weekly or biweekly (every 2 weeks) at a dose that is calculated for each patient according to the manufacturer's instructions taking into account their immunoglobulin trough levels and previous dose of IVIG.

In any embodiment of the above methods, the primary immunodeficiency can be selected from any one of the following types: antibody deficiencies, combined immunodeficiencies, phagocytic cell deficiencies, immune dysregulation, or complement deficiencies. Preferably, the primary immunodeficiency is an antibody deficiency.

In any embodiment of the above methods, the primary immunodeficiency can be selected the group consisting of: X-linked agammaglobulinemia, Common variable immunodeficiency, selective immunoglobulin deficiency, Wiscott-Aldrich syndrome, severe combined immunodeficiency disease (SCID), DiGeorge syndrome, Ataxia-telangectasia, Chronic granulomatous disease, Transient hypogammaglobulinemia of infancy, Agammaglobulinemia, Complement deficiencies, selective IgA deficiency, IL-12 receptor deficiency, IL-12p40 deficiency, IFN-γ receptor deficiencies, STAT1 deficiency, γc deficiency, Jak3 deficiency, RAG 1/2 deficiency, ADA deficiency, X-linked hyper IgM syndrome, MHC class II deficiency, Chediak-Higashi syndrome, defects in early components of classical pathway (C1, C2, C4), defects in early components of alternative pathway (Factor D, Factor P), defects in membrane-attack components (C5-C9), adenosine deaminase deficiency, autoimmune polyendocrinopathy syndrome type 1 (APECED), Bloom syndrome, Cartilage-hair hypoplasia, chronic granulomatous disease, familial atypical mycobacteriosis, hyper immunoglobulin D syndrome, lymphoproliferative disease, X-linked, Nijmogen breakage syndrome, properdin deficiency, purine nucleoside phosphorylase deficiency, X-linked severe combined immunodeficiency, or any other primary immunodeficiency described herein.

In one aspect the present invention provides a computer-implemented method for processing genomic information, the genomic information comprising a subject transcriptome profile, the method comprising:
accessing a reference set of transcriptome profiles of reference subjects, each reference subject either having or not having a primary immunodeficiency (PID);
generating a transcriptomic relationship matrix from the reference set of transcriptome profiles;
fitting the transcriptomic relationship matrix into a linear mixed model to generate a PID prediction equation; and
fitting the subject transcriptome profile to the PID prediction equation.

In another aspect the present invention provides a computer-implemented method for generating a primary immunodeficiency (PID) prediction equation, the method comprising:
accessing a reference set of transcriptome profiles of reference subjects, each reference subject either having or not having a primary immunodeficiency (PID);
generating a transcriptomic relationship matrix from the reference set of transcriptome profiles; and
fitting the transcriptomic relationship matrix into a linear mixed model to generate the PID prediction equation.

In any embodiment of the above methods, further comprises measuring or determining the transcriptome profile of the subject for whom the determination of PID or susceptibility to PID is to be made.

In preferred embodiment of the invention, the linear mixed model is best linear unbiased prediction (BLUP), BayesR, random forest or machine learning approaches, including those as defined herein.

In any embodiment of the above methods, the reference set further comprises a RNA sequence mutation profile.

In any embodiment of the above methods, the method further comprises measuring or determining a RNA sequence mutation profile of the subject for whom the determination of PID or susceptibility to PID is to be made.

In any embodiment of the above methods, the reference set further comprises a DNA sequence mutation profile.

In any embodiment of the above methods, the method further comprises measuring or determining the DNA sequence mutation profile in the subject for whom the determination of PID or susceptibility to PID is to be made. Preferably, the linear mixed model is used to fit the transcriptome profile and the DNA sequence mutation profile of the subject to the PID prediction equation.

In any embodiment of the above methods, the reference set further comprises a metagenome profile and the linear mixed model is used to fit the transcriptome profile and a metagenome profile of the subject to the PID prediction equation.

In a further aspect of the present invention a method determining whether a subject has or is susceptible to developing a primary immunodeficiency (PID), the method comprising using a linear mixed model to fit a metagenomics profile of the subject to a PID prediction equation developed by fitting into a linear mixed model a metagenomic relationship matrix generated from a reference set of metagenomic profiles of reference subjects with and without PID, wherein the prediction equation's result indicates whether the subject has or is susceptible to PID.

In another aspect the present invention provides a non-transitory computer-readable medium storing instructions, which when executed by a processor cause the processor to:
access a reference set of transcriptome profiles of reference subjects, each reference subject either having or not having a primary immunodeficiency (PID);
generate a transcriptomic relationship matrix from the reference set of transcriptome profiles;
fit the transcriptomic relationship matrix into a linear mixed model to generate a PID prediction equation;
receive a subject transcriptome profile; and
fit the subject transcriptome profile to the PID prediction equation.

In another aspect the present invention provides a non-transitory computer-readable medium storing instructions, which when executed by a processor cause the processor to:
access a reference set of transcriptome profiles of reference subjects, each reference subject either having or not having a primary immunodeficiency (PID);
generate a transcriptomic relationship matrix from the reference set of transcriptome profiles; and
fit the transcriptomic relationship matrix into a linear mixed model to generate the PID prediction equation.

In preferred embodiment of the invention, the linear mixed model is best linear unbiased prediction (BLUP), BayesR, or machine learning approaches, including those as defined herein. In a further embodiment of the invention, the machine learning approaches are one of elastic net, ridge regression, lasso regression, random forest, gradient boosting machines, support vector machines, multilayer perceptrons (MLP) or convolutional neural networks (CNN).

In any embodiment of the above a non-transitory computer-readable medium storing instructions, the reference set further comprises a RNA sequence mutation profile.

In any embodiment of the above a non-transitory computer-readable medium storing instructions, the reference set further comprises a DNA sequence mutation profile.

In any embodiment of the above a non-transitory computer-readable medium storing instructions, the reference set further comprises a metagenome profile and the linear mixed model is used to fit the transcriptome profile and a metagenome profile of the subject to the PID prediction equation.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF DRAWINGS

FIG. 1. Schematic overview of the procedure for RNA extraction from blood.

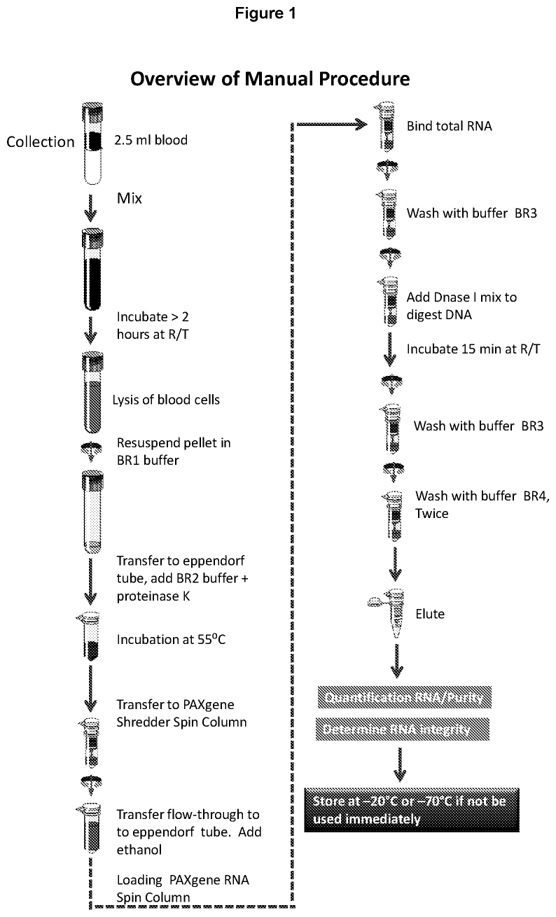

FIG. 2. Schematic overview of the procedure for RNA sequence library generation.

FIG. 3. Differential gene expression analysis comparing 19,521 genes expressed in blood of PID patients and normal matched controls.

FIG. 4. Application of a predictive model using a leave out one prediction approach.

FIG. 5. A receiver operating characteristic (ROC) curve.

FIG. 6. Four individual genes that are differentially, i.e. up or down, regulated in PID.

FIG. 7. An analysis demonstrating examples of significant differences in specific bacterial populations between PID patients and age and sex matched controls generated according to the example of the disclosure.

FIG. 8. Expression of known PID genes in 15 patients (mean±standard deviation). The PID genes shown are from patients included in the study with known mutations.

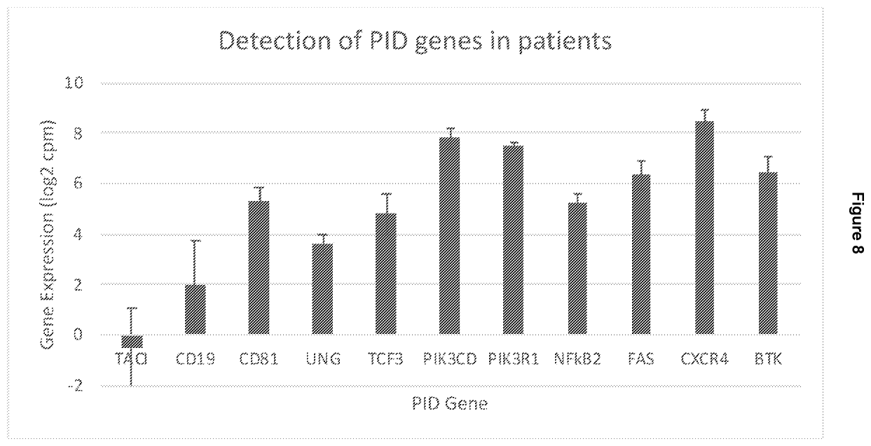

FIG. 9. Mutation detection through whole blood RNAseq. Detection through RNAseq of a dominant missense CXCR4 gene mutation in an allele carried by a PID patient.

FIG. 10. A block diagram of a computer processing system configurable to perform various features of the present disclosure.

### DETAILED DESCRIPTION

A need exists to timely and accurately determine, detect or diagnose PID in a subject. The invention provides such a method that exploits RNAseq, and optionally the metagenome, and a linear mixed model to predict, determine, detect or diagnose PID in a subject.

“Primary immunodeficiency disease” as used herein includes, but is not limited to: combined immunodeficiencies, such as combined immunodeficiency disorders; combined immunodeficiencies with associated or syndromic features, such as congenital thrombocytopenia; predominately antibody deficiencies, such as common variable immunodeficiency disorders; complement deficiencies, such as C1q deficiency; congenital defects of phagocyte number, function, or both, such as severe congenital neutropenias; defects in innate immunity, such as anhidrotic ectodermal dysplasia with immunodeficiency, autoinflammatory disorders, such as familial mediterranean fever; and diseases of immune dysregulation, such as familial hemophagocytic lymphohistiocytosis syndromes.

RNAseq provides at least the following three advantages over DNA analyses.
a) Mutation detection. An advantage for mutation detection in RNA over genomic DNA is that in RNA sequence only expressed genes are represented. The sequence does not include the vast majority of the genome sequence (98%) that is not expressed, thereby reducing the amount of total sequence generation required to identify mutations. This provides for a significant reduction in nucleic acid complexity (and increase in information density for increased throughput and efficiency) particularly if methods for depletion of abundantly expressed globin transcripts are applied before sequencing. The expressed gene sequences in blood are also enriched for expressed immune genes including their coding sequences. As a result less total sequence information needs to be obtained to determine mutation status. This enrichment in RNA of expressed and spliced genes from the genome results in less sequence required to be obtained and therefor lower sequencing costs. The increased relevance and focus of the sequence information obtained from RNA (with the reduced level of irrelevant sequence information) also improves the reliability and efficiency of bioinformatics processing. Genomic sequence approaches for PID recently reported [4] can be used to confirm or complement RNA sequence information.
b) RNA sequencing advantage for measuring PID gene transcript integrity. RNA sequencing has advantages over DNA sequencing in that it can be used to identify RNA structure variants e.g. splicing variants and misplaced intron expression. RNA sequencing can also identify abnormally low expression of a PID gene e.g. in blood as the result of; a transcript defect, destabilising mutation, or hard to identify regulatory region mutations preventing gene expression. The sequence represented in RNA can include coding RNAs and non-coding RNAs. Short read NGS technologies are well suited to this, however, long-read sequencing technologies, such as Pacific-Biosciences (PacBio) SMRT and Oxford Nanopore are suitable and have advantages for measuring transcript presence and integrity.
c) RNA sequencing advantage for measuring immune cell composition and activity. In addition to having advantages over DNA sequencing for mutation detection as a component of PID determination, detection or diagnosis, RNA sequencing provides functional information (not contained in a DNA sequence) as it includes a comprehensive measure of gene activities, in this case the activity of genes in immune cells in blood. A holistic analysis of gene expression levels can assist in identifying immune deficiency, since expression of many genes measured in blood or blood derived cells can identify deficient or abnormal gene expression concomitant with immune cell population and immune cell function changes. The inability of PID patients to fight infections are a direct result of such immune cell population and immune cell functional changes in blood, and these changes are expected to be evident in the RNA transcript profile.

Due to the involvement of different cell types, and the large number of immune genes subsequently or secondarily affected in deficiency, a whole transcriptome approach with an encompassing mixed model analysis such as Best Linear Unbiased Prediction (BLUP) or BayesR [5] is required, modified to use read counts or transformed read counts rather than SNP information (a straight forward modification). BLUP or BayesR is required to assess the full extent of distinguishing characteristics of PID patients. The immune function information provided in one step by RNA sequencing (if the information is captured with appropriate analysis) provides an advantage in cost, time, and resolution over combinations of immunological status assays usually required for PID determination, detection or diagnosis such as lymphocyte proliferation and cytotoxicity assays, flow cytometry, measurement of serum immunoglobulin levels, complete blood cell counts, neutrophil function tests, and complement assays.

RNAseq is used in disease research as it is useful for investigative purposes, but due to a number of difficulties RNAseq is not used in a clinical setting for determination, detection or diagnostic purposes or for the routine assessment of diseases [1]. Difficulties in being able to use whole transcriptome RNA expression information stem from the complexity of the information (data represented for 1000s of genes) and a lack of knowledge of the relevant components of the information (such as specific genes and pathways) to monitor expression levels of for determination, detection or diagnosis of diseases such as PID. Further to that, a lack of suitable statistical analysis approaches exists to identify and utilise putative mRNA biomarkers. Even when mRNA biomarkers in RNAseq data can be identified, a lack of standardisation for RNA sequence processing and defined statistical analysis limits potential clinical application.

More developed approaches exist for DNA sequencing, providing a more established paths and standards for mutation detection to complement clinical immune system information. RNA-sequencing for mutation detection in expressed gene sequences is useful, however, functional information that may be provided by the transcriptome sampling can be also be used. A BLUP or BayesR linear mixed model approach provides an analysis of the transcript abundance information in RNASeq data that permits it to be used directly as a diagnostic. Limitations of using RNA-sequencing alone w/o RNA expression BLUP or BayesR analysis are that mutations can be detected in expressed gene sequences, however functional information that may be provided by the RNA sequence profile/transcriptome data on the immune system is not fully captured and used.

A BLUP or BayesR model provides an approach that permits very many effects including small effects in cells and pathways (stemming from a deficiency in the immune system) to be incorporated into analysis and assessment for diagnosis. This approach may obviate the need for clinical immunological tests as it can capture a broad range of functional consequences at the RNA level. An approach taken for diagnosis discovery (not using BayesR or BLUP) would typically be to try and identify key genes (in addition to PID genes) as markers of function that could be used in place of clinical immunological tests. For example, cell composition changes in PID could be assessed by measuring transcripts for specific markers such as CD4, CD14, CD3, CD56, and CD19. Similarly other specific pathways or gene networks found to be affected in PID could also be used either as individual tests, combined tests, or by deriving individual gene set information from RNAseq data. BLUP and BayesR provide a solution as they can be applied directly utilising complete RNAseq information, and thereby incorporate large numbers of genes affected in the analysis, and can measure large numbers of small effects expected to occur as a result of PID mutations.

BLUP and BayesR approaches proposed by the inventors, have an advantage over other more targeted diagnostic marker approaches, as they use full information from the gene expression profiles (using all genes expressed in blood in the analysis) directly as the diagnostic signature, as opposed to using a single or more limited number of informative and/or known markers (if they were discovered and available to use for PID diagnosis application) as separate gene expression assays or deriving specific information from RNAseq data. In addition, the BLUP or BayesR approach is straightforward and efficient, requiring a single computational step without human intervention or requiring a combination of analysis methods. The transcriptomic BLUP or BayesR approach is also best suited to being able to identify a range of overlapping immune deficiency gene expression patterns reflecting disease from a diversity of causal mutations in different patients. A more limited set of diagnostic gene markers (if they were available) may not be able to identify a range of PID disease diversity. In addition, when trained on appropriate affected and non-affected patient reference profiles, the BLUP/BayesR approaches do not require specific knowledge of all aspects of the functional changes being measured for diagnosis in order to be implemented effectively, and therefore are able to capture informative consequences of mutations not yet understood to assist in the diagnosis.

The inventors have overcome difficulties by providing a sequencing and whole transcriptomic BLUP/BayesR methodology to determine, detect or diagnose PID. This obviates the need for the functional tests required for PID diagnosis by providing a method of contemporaneously assaying genomic information and immune cell function in one-step by molecular means. Avenues of investigation for improved functional tests mostly include expanding the cell types being examined using antibody markers and FACS, and examination of cells for defective function tested under activation conditions.

RNAseq is not contemplated as a diagnostic, but used as an investigative tool to identify genes and pathways associated with immune function. In this case, investigators would start by selecting certain genes from various analyses as candidates for immune function monitoring and diagnosis. For example, taking parallels from RNAseq application taken in other diseases, PID subject samples and the normal subject samples would potentially be compared by various means, and differentially expressed transcripts will be identified as different between the PID subjects' and normal subjects' samples. Gene ontology enrichment analysis would be performed using tools such as the DAVID website (https://david.ncifcrf.gov/). The differential gene expression profile could also be subjected to gene set enrichment analysis using gene set enrichment analysis (GSEA) with MSigDB public immunological gene signatures. Investigators are likely to perform RNAseq for investigative purposes and search in RNAseq data for known genes and pathways, or known cell markers, typically conducting RNAseq on subsets of blood cells. A BLUP approach on RNAseq from whole blood is able to incorporate information from known, and unknown gene networks that are not well understood, where direct and indirect effects can be captured has not been envisaged as a direct diagnostic and not as a surrogate for a range of cell-based assays. Nowhere is it suggested that whole blood transcriptomic BLUP be used directly as a diagnostic to replace cell and immune function assays including for PID.

BLUP has been used to classify samples into subsets to aid in investigative studies, and enhance genetic diagnosis (SNP variance) for multi-genic diseases. In some cases, BLUP can be used to combine diverse types of clinical information to provide more accurate prognosis. Application of BLUP for disease classification has been applied in neuroblastoma [6].

Other clinical information can be used in combination with information from the RNA-based methods described above to assist in diagnosis, including microbial colonisation information. Recording and managing infections including in some cases microbial diagnostic approaches for organisms that are pathogenic are an important component of PID diagnosis.

Metagenomic sequencing extends the analysis of microbial compositions beyond pathogens with information that includes a comprehensive measure of microbial community activities. A holistic analysis of microbial interface management will be able to assist in identifying immune deficiency, since the presence of many organisms in mucosa or hair follicles can identify deficient, or abnormal community structures, or combinations of specific organisms concomitant with immune cell population and immune cell function changes.

As used herein, “RNAseq” or “transcriptome” refers to genes expressed and then sequenced, the sequence reads of which align to exon sequences in the genome or a reference transcriptome database. A “transcriptome profile” is the vector of counts of the sequence reads, and accordingly, is the overall, characterizing composition of genes expressed in a sample.

A transcriptomic relationship matrix may be generated from transcriptome profiles as set out in the examples, and may be generated as part of the method of the invention or may be pre-existing.

In one embodiment of the present invention, the linear mixed model is BLUP or BayesR. As used herein, a “linear mixed model”, also called a “multilevel model” or a “hierarchical mode”, refers to a class of regression models that takes into account both the variation that is explained by the independent variables of interest and the variation that is not explained by the independent variable of interest, or random effects. Examples of linear mixed models include, but are not limited to, BayesR and best linear unbiased prediction (BLUP). The person skilled in the art will be aware of other appropriate linear mixed models.

In one embodiment, the PID prediction equation is any one described herein, including the Example.

The predictive scores (either relative or absolute) generated may be used to classify subjects into high (e.g. a score where a higher value is high risk) or low (e.g. a score where a lower value is low risk) risk of having PID. For example, when using an absolute predictive score, a score of >0.2 provides a diagnostic assay for detection of PD with a sensitivity of 93% and a specificity of 47%. A score of >0.4 provides a diagnostic assay for detection of PID with a sensitivity of 73% and a specificity of 73%. A score of >0.6 provides a diagnostic assay for detection of PID with a sensitivity of 53% and a specificity of 100%. In contrast, for example, when using a relative predictive score (whereby a relative predictive score for each patient when matched with a control group is determined by subtracting the healthy control score from the patient score), a score of −0, >0.1 and >0.2 provides a diagnostic assay for detection of PID with a sensitivity of 93%, 80% and 73% respectively.

In one embodiment of the present invention, the reference set further comprises an RNA sequence mutation profile. In a further embodiment of the present invention, the reference set further comprises an RNA sequence mutation profile and the linear mixed model is used to fit the transcriptome profile and a RNA sequence mutation profile of the subject to the PID prediction equation.

In one embodiment of the present invention, the reference set further comprises a DNA sequence mutation profile. In a further embodiment of the present invention, the reference set further comprises a DNA sequence mutation profile and the linear mixed model is used to fit the transcriptome profile and a DNA sequence mutation profile of the subject to the PID prediction equation.

In one embodiment of the present invention, the reference set further comprises a metagenome profile. In a further embodiment of the present invention, the reference set further comprises a metagenome profile and the linear mixed model is used to fit the transcriptome profile and a metagenome profile of the subject to the PID prediction equation.

The term “Metagenome” as used herein refers to the total DNA recovered from a sample, including the DNA from microbial inhabitants or the “microbiome” of the sample. “Metagenome profile” as used herein refers to the overall, characterizing composition of microbial DNA in a sample. “Microbiome” as used herein refers to all of the microbes in a sample.

In one embodiment of the method of the present invention, the metagenome profile is obtained from a mouth swab, nose swab, throat swab, saliva, faeces, skin, or a hair follicle. That is, the metagenome profile is obtained from a sample that comprises the microbiome from a mouth swab, nose swab, throat swab, saliva, faecal sample, skin sample or hair follicle sample.

The term “Gene sequence mutation” as used herein encompasses both an RNA sequence mutation and a DNA sequence mutation, and refers to a change from the wild-type or a reference sequence of one or more nucleic acid molecules. “Mutations” include without limitation, base pair substitutions, additions and deletions of at least one nucleotide from a nucleic acid molecule of known sequence. A mutated nucleic acid can be expressed from or found on one allele (heterozygous) or both alleles (homozygous) of a gene, and may be somatic or germ line. Accordingly, a “gene sequence mutation profile” is the overall, characterizing composition of gene sequence mutations in a sample.

A gene sequence mutation also encompasses:
a) where the RNA sequence of a PID gene is shown to have such a known mutation resulting in PID;
b) where a new mutation (e.g. a missense mutation resulting in an amino acid change or nonsense mutation resulting a frameshift) affecting a predicted structure or function of a protein is detected in a known PID gene from the RNA sequence;
c) where a dominant mutation is detected in one allele from the RNA sequences;
d) where two different mutations occur in the same gene, but on two different alleles;
e) where a known mutation in RNA is inferred or imputed from linkage to a co-occurring haplotype marker in the RNA expressed from the same gene, or nearby gene on the chromosome;
f) where expression of a PID gene sequence normally expressed in blood is not detected in blood RNA (indicating a serious regulatory defect or destabilising mutation);
g) where the exon structure of the mutated PID gene determined by RNAseq is defective (indicating a splicing defect);
h) where one or more (1-3) additional PID gene mutations are detected in the same patient from the RNA/cDNA sequence; and
i) where the sequence of several other genes, or imputed sequence of other genes, detected in the RNA profile contributes to PID severity.

Expressed differently, in one embodiment of the method of the present invention, the mutation profile comprises:
a) a RNA sequence of a PID gene comprising a known mutation resulting in PID;
b) a new mutation, optionally a frameshift mutation, that affects structure or function of a protein encoded by a known gene mutation of which results in PID;
c) a dominant mutation in one allele that results in PID;
d) two different mutations in the same gene, but on two different alleles that result in PID;
e) a known mutation in RNA that is inferred or imputed by linkage to a co-occurring marker for a mutation resulting in PID;
f) absence of expression of a gene normally expressed in non-PID subjects indicating a regulatory defect or destabilising mutation;
g) a defective exon structure indicating a splicing defect;
h) one or more, optionally one to three, additional mutations resulting in PID; or
i) a sequence of more than one other gene, or an imputed sequence of more than one other gene, that contributes to PID severity.

As used herein, “reference set” or “training set” refers to a group of transcriptome profiles, gene sequence mutation profiles, or metagenome profiles obtained from subjects with and without PID, i.e. “reference subjects”, used to generate a transcriptomic relationship matrix, subsequently used to predict PID.

The term “marker” or “biomarker” as used herein refers to a biochemical, genetic (either DNA or RNA), or molecular characteristic that is a surrogate for and therefore indicative/predictive of a second characteristic, for example a genotype, phenotype, pathological state, disease or condition.

In one embodiment of the present invention, the transcriptome profile or sequence mutation profile is obtained from sputum, blood, amniotic fluid, plasma, semen, bone marrow, tissue, urine, peritoneal fluid, or pleural fluid, optionally obtained by fine needle biopsy. In a further embodiment, the blood comprises peripheral blood mononuclear cells.

A “subject” as used herein may be human or a non-human animal, for example a domestic, a zoo, or a companion animal. In one embodiment, the subject is a mammal. The mammal may be an ungulate and/or may be equine, bovine, ovine, canine, or feline, for example. In one embodiment, the subject is a primate. In one embodiment, the subject is human. Accordingly, the present invention has human medical applications, and also veterinary and animal husbandry applications, including treatment of domestic animals such as horses, cattle and sheep, and companion animals such as dogs and cats.

Throughout the description and claims of this specification, the word “comprise” and variations of the word, such as “comprising” and “comprises”, means “including but not limited to”, and is not intended to exclude other additives, components integers or steps.

As used herein, “determining whether a subject has or is susceptible to developing a PID” refers to detecting or diagnosing a PID in a subject, or predicting or prognosing, that a subject is likely to develop a PID. The invention also encompasses detecting a PID in a subject or detecting susceptibility to a PID in a subject. In other words, the invention encompasses determining, detecting or diagnosing a PID in a subject and/or determining, detecting or diagnosing susceptibility to a PID in a subject.

The term “biological sample” as used herein refers to a sample which may be tested for a particular “gene expression profile”, “gene sequence mutation profile”, “transcriptome profile” or “sequence mutation profile” (where the sequence mutation profile may be mutation in RNA and/or DNA). A sample may be obtained from an organism (e.g. a human patient) or from components (e.g. cells) of an organism. The sample may be of any relevant biological tissue or fluid which comprises RNA and/or DNA. The sample may be a “clinical sample” which is a sample derived from a patient. Such samples include, but are not limited to, sputum, blood, blood cells (e.g. white cells), amniotic fluid, plasma, semen, bone marrow, and tissue or fine needle biopsy samples, urine, peritoneal fluid, and pleural fluid, or cells therefrom. Biological samples may also include sections of tissues such as frozen sections taken for histological purposes. A biological sample may also be referred to as a “patient sample”. In one embodiment, the method of the invention is not practiced on a human or animal body, for example, the test profile may be determined by analysing previously obtained biological sample.

The term “gene” as used herein refers to a nucleic acid sequence that comprises coding sequences necessary for producing a polypeptide or precursor. Control sequences that direct and/or control expression of the coding sequences may also be encompassed by the term “gene” in some instances. The polypeptide or precursor may be encoded by a full-length coding sequence or by a portion of the coding sequence. A gene may contain one or more modifications in either the coding or the untranslated regions that could affect the biological activity or the chemical structure of the polypeptide or precursor, the rate of expression, or the manner of expression control. Such modifications include, but are not limited to, mutations, insertions, deletions, and substitutions of one or more nucleotides, including single nucleotide polymorphisms that occur naturally in the population. The gene may constitute an uninterrupted coding sequence or it may include one or more subsequences.

The term “gene expression level” or “expression level” as used herein refers to the amount of a “gene expression product” or “gene product” in a sample. “Gene expression profile” or “gene expression signature” as used herein refers to a group of “gene expression products” or “gene products” produced by a particular cell or tissue type wherein expression of the genes taken together, or the differential expression of such genes, is indicative and/or predictive of a pathological state, disease or condition, such as an immune disorder. A “gene expression profile” can be either qualitative (e.g. presence or absence) or quantitative (e.g. levels or mRNA copy numbers). Thus, a “gene expression profile” can also be used to determine the numbers of specific cell types in a heterogeneous sample of cells, such as the number of T cells in a blood sample, based on the amount of cell-type specific “gene expression products” or “gene products”.

The term “gene expression product” or “gene product” as used herein refers to the RNA transcription products (RNA transcript) of a gene, including mRNA, and the polypeptide translation product of such RNA transcripts. A “gene expression product” or “gene product” can be, for example, a polynucleotide gene expression product (e.g. an un-spliced RNA, an mRNA, a splice variant mRNA, a microRNA, a fragmented RNA) or a protein expression product (e.g. a mature polypeptide, a splice variant polypeptide).

The term “immune cell” as used herein refers to cells, such as lymphocytes, including natural killer cells, T cells, B cells, macrophages and monocytes, dendritic cells or any other cell which is capable of producing an “immune effector molecule” in response to direct or indirect antigen stimulation. The term “immune effector molecules” are molecules which are produced in response to cell activation or stimulation by an antigen, including, but not limited to, cytokines such as interferons (IFN), interleukins (IL), such as IL-2, IL-4, IL-10 or IL-12, tumor necrosis factor alpha (TNF-α), colony stimulating factors (CSF), such as granulocyte (G)-CSF or granulocyte macrophage (GM)-CSF, complement and components in the complement pathway.

The term “immune disorder” as used herein refers to a pathological state, disease or condition characterized by a dysfunction in the immune system. “Immune disorders” include, but are not limited to, autoimmune disorders, such as scleroderma, allergies, such as allergic rhinitis, and immunodeficiencies, such as primary immunodeficiency disease.

The term “normal immune system” as used herein refers to an immune system that has a normal composition of immune cells and wherein said immune cells are not dysfunctional. A “normal” or “healthy” subject as used herein refers to a subject with a “normal immune system”.

The term “nucleic acid” as used herein refers to DNA molecules (e.g. cDNA or genomic DNA), RNA molecules (e.g. mRNA), DNA-RNA hybrids, and analogs of the DNA or RNA generated using nucleotide analogs. The nucleic acid molecule can be a nucleotide, oligonucleotide, double-stranded DNA, single-stranded DNA, multi-stranded DNA, complementary DNA, genomic DNA, non-coding DNA, messenger RNA (mRNA), microRNA (miRNA), small nucleolar RNA (snoRNA), ribosomal RNA (rRNA), transfer RNA (tRNA), small interfering RNA (siRNA), heterogeneous nuclear RNAs (hnRNA), or small hairpin RNA (shRNA).

A method of the present invention may comprise the further step of treating a PID in a subject determined to have or be susceptible to a PID.

Accordingly, also disclosed is treatment of a PID in a subject determined to have or be susceptible to a PID by a method of the invention.

Accordingly, disclosed herein is a method of treating a PID in a subject, the method comprising:
administering to the subject an antibiotic, an immunoglobulin, an interferon, a growth factor, gene therapy, or enzyme replacement therapy; or
transplanting a hematopoietic stem cell into the subject,
wherein the subject is determined to have or be susceptible to developing PID by the method of the present invention.

Also disclosed is use of an antibiotic, an immunoglobulin, an interferon, a growth factor, an enzyme, a gene, or a hematopoietic stem cell in the manufacture of a medicament for treating a PID in a subject, wherein the subject is determined to have or be susceptible to developing PID by the method of the present invention.

Also disclosed is an antibiotic, an immunoglobulin, an interferon, a growth factor, an enzyme, a gene, or a hematopoietic stem cell for use in a method of treating PID in a subject, wherein the subject is determined to have or be susceptible to developing PID by the method of the present invention.

For PID determination, detection or diagnosis, RNAseq provides three main advantages over DNA sequencing for mutation detection and immune function assessment: (a) mutations are detected in expressed genes only; (b) PID gene transcript integrity; (c) immune cell composition and activity.

In one embodiment, the PID to be treated is selected from: combined immunodeficiencies, such as combined immunodeficiency disorders; combined immunodeficiencies with associated or syndromic features, such as congenital thrombocytopenia; predominately antibody deficiencies, such as common variable immunodeficiency disorders; complement deficiencies, such as C1q deficiency; congenital defects of phagocyte number, function, or both, such as severe congenital neutropenias; defects in innate immunity, such as anhidrotic ectodermal dysplasia with immunodeficiency, autoinflammatory disorders, such as familial mediterranean fever; and diseases of immune dysregulation, such as familial hemophagocytic lymphohistiocytosis syndromes.

Effective treatments of PIDs include managing infection, boosting the immune system, hematopoietic stem cell transplantation, gene therapy, and enzyme replacement therapy.

Managing Infections Includes:
treating infections with antibiotics, usually rapidly and aggressively—infections that do not respond may require hospitalization and intravenous (IV) antibiotics.
preventing infections, for example with long-term antibiotic treatment to prevent respiratory infections and associated permanent damage to the lungs and ears, and avoidance of vaccinating children with PID using vaccines containing live viruses, such as oral polio and measles-mumps-rubella.
treating symptoms using pharmaceutical substances such as ibuprofen for pain and fever, decongestants for sinus congestion, expectorants to thin mucus in the airways, or using postural drainage in which gravity and light blows are applied to the chest to clear the lungs.

Boosting the Immune System Includes:
immunoglobulin therapy, usually intravenously every few weeks or subcutaneously once or twice a week.
gamma interferon therapy to combat viruses and stimulate immune cells, usually intramuscularly three times a week, most often to treat chronic granulomatous disease.
growth factor therapy to increase the levels of white blood cells.

Stem cell transplantation offers a permanent cure for several forms of life-threatening PID.

It will be appreciated by the person skilled in the art that the exact manner of administering to a subject a therapeutically effective amount of an antibiotic, an immunoglobulin, an interferon, a growth factor, hematopoietic stem cells, a gene for gene therapy, or an enzyme for enzyme replacement therapy will be at the discretion of the medical practitioner with reference to the PID to be treated or prevented. The mode of administration, including dosage, combination with other agents, timing and frequency of administration, and the like, may be affected by the subject's likely responsiveness to treatment, as well as the subject's condition and history.

The antibiotic, immunoglobulin, interferon, growth factor, hematopoietic stem cells, gene for gene therapy, or enzyme for enzyme replacement therapy will be formulated, dosed, and administered in a fashion consistent with good medical practice. Factors for consideration in this context include the particular PID being treated or prevented, the particular subject being treated, the clinical status of the subject, the site of administration, the method of administration, the scheduling of administration, possible side-effects and other factors known to medical practitioners. The therapeutically effective amount of antibiotic, immunoglobulin, interferon, growth factor, hematopoietic stem cells, gene for gene therapy, or enzyme for enzyme replacement therapy to be administered will be governed by such considerations.

The antibiotic, immunoglobulin, interferon, growth factor, hematopoietic stem cells, gene for gene therapy, or enzyme for enzyme replacement therapy may be administered systemically or peripherally, for example by routes including intravenous (IV), intra-arterial, intramuscular (IM), intraperitoneal, intracerobrospinal, subcutaneous (SC), intra-articular, intrasynovial, intrathecal, intracoronary, transendocardial, surgical implantation, topical and inhalation (e.g. intrapulmonary).

The term “therapeutically effective amount” refers to an amount of antibiotic, immunoglobulin, interferon, growth factor, hematopoietic stem cells, gene for gene therapy, or enzyme for enzyme replacement therapy effective to treat a PID in a subject.

The terms “treat”, “treating” or “treatment” refer to both therapeutic treatment and prophylactic or preventative measures, wherein the aim is to prevent or ameliorate a PID in a subject or slow down (lessen) progression of a PID in a subject. Subjects in need of treatment include those already with the PID as well as those in which the PID is to be prevented.

The terms “preventing”, “prevention”, “preventative” or “prophylactic” refers to keeping from occurring, or to hinder, defend from, or protect from the occurrence of a PID, including an abnormality or symptom. A subject in need of prevention may be prone to develop the PID.

The term “ameliorate” or “amelioration” refers to a decrease, reduction or elimination of a PID, including an abnormality or symptom. A subject in need of amelioration may already have the PID, or may be prone to develop the PID, or may be in whom the PID is to be prevented.

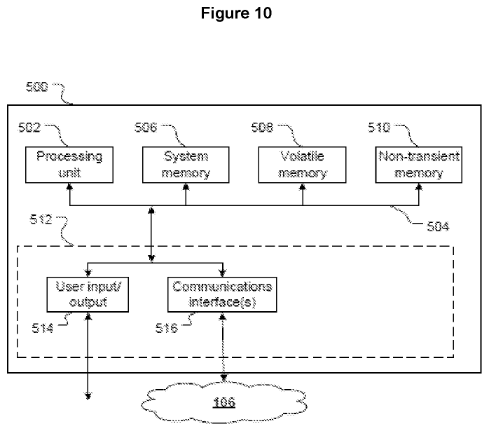

FIG. 10 provides a block diagram of a computer processing system 500 configurable to implement embodiments and/or features described herein. System 500 is a general purpose computer processing system. It will be appreciated that FIG. 10 does not illustrate all functional or physical components of a computer processing system. For example, no power supply or power supply interface has been depicted, however system 500 will either carry a power supply or be configured for connection to a power supply (or both). It will also be appreciated that the particular type of computer processing system will determine the appropriate hardware and architecture, and alternative computer processing systems suitable for implementing features of the present disclosure may have additional, alternative, or fewer components than those depicted.

Computer processing system 500 includes at least one processing unit 502—for example a general or central processing unit, a graphics processing unit, or an alternative computational device). Computer processing system 500 may include a plurality of computer processing units. In some instances, where a computer processing system 500 is described as performing an operation or function all processing required to perform that operation or function will be performed by processing unit 502. In other instances, processing required to perform that operation or function may also be performed by remote processing devices accessible to and useable by (either in a shared or dedicated manner) system 500.

Through a communications bus 504, processing unit 502 is in data communication with a one or more computer readable storage devices which store instructions and/or data for controlling operation of the processing system 500. In this example system 500 includes a system memory 506 (e.g. a BIOS), volatile memory 508 (e.g. random access memory such as one or more DRAM modules), and non-volatile (or non-transitory) memory 510 (e.g. one or more hard disk or solid state drives). Such memory devices may also be referred to as computer readable storage media.

System 500 also includes one or more interfaces, indicated generally by 512, via which system 500 interfaces with various devices and/or networks. Generally speaking, other devices may be integral with system 500, or may be separate. Where a device is separate from system 500, connection between the device and system 500 may be via wired or wireless hardware and communication protocols, and may be a direct or an indirect (e.g. networked) connection.

Wired connection with other devices/networks may be by any appropriate standard or proprietary hardware and connectivity protocols, for example Universal Serial Bus (USB), eSATA, Thunderbolt, Ethernet, HDMI, and/or any other wired connection hardware/connectivity protocol.

Wireless connection with other devices/networks may similarly be by any appropriate standard or proprietary hardware and communications protocols, for example infrared, BlueTooth, WiFi; near field communications (NFC); Global System for Mobile Communications (GSM), Enhanced Data GSM Environment (EDGE), long term evolution (LTE), code division multiple access (CDMA—and/or variants thereof), and/or any other wireless hardware/connectivity protocol.

Generally speaking, and depending on the particular system in question, devices to which system 500 connects—whether by wired or wireless means—include one or more input/output devices (indicated generally by input/output device interface 514). Input devices are used to input data into system 100 for processing by the processing unit 502. Output devices allow data to be output by system 500. Example input/output devices are described below, however it will be appreciated that not all computer processing systems will include all mentioned devices, and that additional and alternative devices to those mentioned may well be used.

For example, system 500 may include or connect to one or more input devices by which information/data is input into (received by) system 500. Such input devices may include keyboards, mice, trackpads (and/or other touch/contact sensing devices, including touch screen displays), microphones, accelerometers, proximity sensors, GPS devices, touch sensors, and/or other input devices. System 500 may also include or connect to one or more output devices controlled by system 500 to output information. Such output devices may include devices such as displays (e.g. cathode ray tube displays, liquid crystal displays, light emitting diode displays, plasma displays, touch screen displays), speakers, vibration modules, light emitting diodes/other lights, and other output devices. System 500 may also include or connect to devices which may act as both input and output devices, for example memory devices/computer readable media (e.g. hard drives, solid state drives, disk drives, compact flash cards, SD cards, and other memory/computer readable media devices) which system 500 can read data from and/or write data to, and touch screen displays which can both display (output) data and receive touch signals (input).

System 500 also includes one or more communications interfaces 516 for communication with a network, such as the Internet in environment 100. Via a communications interface 516 system 500 can communicate data to and receive data from networked devices, which may themselves be other computer processing systems.

System 500 stores or has access to computer applications (also referred to as software or programs)—i.e. computer readable instructions and data which, when executed by the processing unit 502, configure system 500 to receive, process, and output data. Instructions and data can be stored on non-transitory computer readable medium accessible to system 500. For example, instructions and data may be stored on non-transitory memory 510. Instructions and data may be transmitted to/received by system 500 via a data signal in a transmission channel enabled (for example) by a wired or wireless network connection over interface such as 512.

Applications accessible to system 500 will typically include an operating system application such as Microsoft Windows®, Apple OSX, Apple 10S, Android, Unix, or Linux.

In some cases part or all of a given computer-implemented method will be performed by system 500 itself, while in other cases processing may be performed by other devices in data communication with system 500.

The transcriptome differences form a genetic signature for the disease can be identified using a learning software algorithm and proprietary reference database.

The genomic algorithm generates a predictive score which can be used to identify patients with disease. A component of the software is comprised of a bioinformatic pipeline that searches for specific gene mutations which have previously established to be causative of PID.

The present invention includes methods described herein and a program that can utilise a set of existing bioinformatics tools (including R for the transcriptomic relationship matrix and BLUP prediction, and GATK) for mutation detection causative of PID.

The invention will now be described with reference to the following, non-limiting examples.

### Example

Un-blinded, ex vivo, study using samples from subjects with confirmed primary immunodeficiency disease and normal subjects.

Study Outline

An un-blinded, multi-centred, ex vivo, study using biological samples collected from 20 subjects with confirmed primary immunodeficiency disease (PID) and 20 normal subjects.

The study demonstrated that PID may be diagnosed using:
(i) gene expression data, i.e. RNAseq or the transcriptome, obtained by RNA sequencing;
(ii) gene expression data, i.e. RNAseq or the transcriptome, combined with gene sequence data;
(iii) gene expression data, i.e. RNAseq or the transcriptome, combined with microbial metagenome data, obtained by targeted or untargeted massively parallel sequencing and using a linear mixed model prediction approach can alone be used to diagnose PID; or
(iv) microbial metagenome data,
obtained by targeted or untargeted massively parallel sequencing and using a linear mixed model prediction.

Exclusion Criteria

Subjects who have previously undergone a haematopoietic stem cell transplant were excluded from the study.

Sample Collection

Blood cells from peripheral venous whole blood were collected for RNA extraction. Microbial samples were collected from mouth buccal swab, nose swab, throat swab, saliva, faecal sample, skin sample or hair follicle sample for DNA extraction.

Transcriptome Profile Determination

RNA sequencing was performed to identify gene sequence mutations indicative of PID, and to determine the gene expression profile of PID subjects for comparison to normal subjects.

i) Sample Collection and RNA Extraction

Blood cells from peripheral venous whole blood were prepared using PAXgene™ blood RNA tubes (PAXgene Blood RNA Kit (50)-Cat No./ID: 762164) according to the manufacturer's instructions. The reagent composition of PAXgene Blood RNA Tubes protects RNA molecules from degradation and can stabilise cellular RNA of human whole blood up to 3 days at 18-25° C. or up to 5 days at 2-8° C. or at 8 years at −20° C./−70° C.

2.5 ml of drawn blood was collected into PAXgene blood RNA tubes and incubated for at least 2 hours at room temperature to ensure complete lysis of blood cells. If the PAXgene Blood RNA Tube was stored at 2-8° C., −20° C. or −70° C. after blood collection, the sample is first equilibrated to room temperature, and then stored at room temperature for 2 hours before starting the procedure. After preparing buffers, the following steps were taken:
(i) Centrifuge the PAXgene Blood RNA Tube for 10 minutes at 3000-5000×g using a swing-out rotor and remove the supernatant.
(ii) Add 4 ml RNase-free water to the tube and close it using a fresh BD Hemogard Closure supplied with the kit.
(iii) Vortex until the pellet is visibly dissolved. Centrifuge for 10 minutes at 3000-5000×g using a swing-out rotor and remove the supernatant completely.
(iv) Add 350 μl Buffer BR1 and vortex until the pellet is visibly dissolved.
(v) Remove the sample into a 1.5 ml eppendorf tube. Successively add 300 μl buffer BR2 and 40 μl proteinase K. Mix by vortexing for seconds.
(vi) Incubate for 10 minutes at 55° C. using a shaker—incubator at 400-1400 rpm.
(vii) Pipet the lysate directly into a PAXgene Shredder spin column (lilac) placed in a 2 ml collecting tube and centrifuge for 3 minutes at maximum speed (but not to exceed 20 000×g, which may damage the columns).
(viii) Carefully transfer the entire supernatant of the flow-through fraction to a fresh 1.5 ml tube without disturbing the pellet in the processing tube.
(ix) Add 350 μl ethanol (96-100%, purity grade p.a.). Mix by vortexing and centrifuge briefly to remove drops from the inside of the tube lid.
(x) Pipet 700 μl into the PAXgene RNA spin column (red) placed in a 2 ml processing tube and centrifuge for 1 minute at 16000×g (8000-20,000×g). Discard the flow-through.
(xi) Pipette the remaining sample into the PAXgene RNA spin column and centrifuge for 1 minute at 16000×g (8000-20,000×g). Discard flow-through.
(xii) Wash the column with 350 μl Buffer BR3 into. Centrifuge for 1 minute at 16 000×g (8000-20 000×g).
(xiii) Add 80 μl DNase I mix (80 μl) directly onto the centre of PAXgene RNA spin column membrane and incubate at room temperature (20-30° C.) for 15 minutes.
(xiv) Pipet 350 μl Buffer BR3 into the PAXgene RNA spin column and centrifuge for 1 minute at 16 000×g (8000-20 000×g). Discard flow-through.
(xv) Wash the column with 500 μl BR4 and centrifuge for 1 minute at 16 000×g (8000-20 000×g). Discard the flow-through and centrifuge for another 1 minute at 16 000×g (8000-20 000×g).
(xvi) Add another 500 μl Buffer BR4 to the column and centrifuge for 3 minutes at 16 000×g (8000-20 000×g). Discard the processing tube containing the flow-through, and place the PAXgene RNA spin column in a new 2 ml processing tube. Centrifuge for 2 minute at 16 000×g (8000-20 000×g). Transfer the column in a 1.5 ml tube.
(xvii) Add 40 μl Buffer BR5 directly onto the column membrane. To elute RNA by centrifuging for 2 min at 16 000×g (8000-20 000×g). (Note: It is important to the centre of PAXgene RNA spin column for wetting the entire membrane with Buffer BR5 in order to achieve maximum elution efficiency.)
(xviii) Quantitate RNA/Purity, e.g. using NanaDrop 1000/2000 or Qubit instruments and using RNA specific binding fluorescent dye such as Quant-iT™ RNA.
(xix) Determine RNA integrity, e.g. using a BioAnalyser 2100 or TapeStation 2200 instrument (Agilent Technologies)
(xx) If the RNA samples will not be used immediately, store at −20° C. or −70° C.

ii) RNA Sequencing

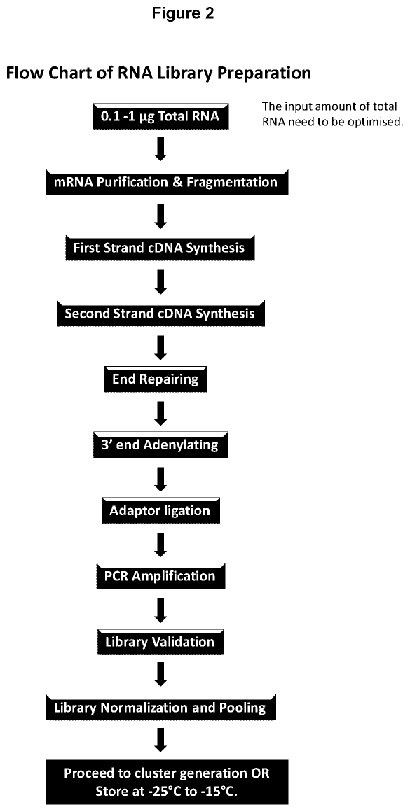

RNAseq libraries were prepared using the TruSeq RNA sample preparation kit (Illumina) according to the manufacturer's protocol outlined in FIG. 2.

Preparation of the whole transcriptome sequencing library was conducted using Illumina's “TruSeq Stranded Total RNA Library Prep Kit with Ribo-Zero Globin Set” according to manufacturer's instructions.

Multiplexes of libraries each with one of 12 indexed adaptors, were pooled. Each pool was sequenced on one flowcell lane on the HiSeq2000 sequencer (Illumina) in a 101 cycle paired end run.

iii) Gene Expression Profile Generation and Sequence Analysis

100 base long paired end-reads generated by the HiSeq2000 sequencer (Illumina) were called with CASAVA v1.8 and output in fastq format. Sequence quality was assessed using trimmomatic (v0.39) and scripts were used to trim and filter poor quality bases and sequence reads. Bases with quality score less than 20 were trimmed from the 3′ end of reads. Reads with mean quality score less than 20, or greater than 3 N, or final length less than 35 bases were discarded. Only paired reads were retained for alignment.

After RNA-sequencing use of the Trimmomatic software [7], raw read sequences were trimmed for minimum quality at the 3′ end (phred score of at least 30), cleaned of adapter traces and filtered for a final minimum length of 32 bp. Alignment to the Ensembl GRCh38.84 was performed using hisat2 (v2.1) or alternatively UCSC hg19 reference genome (Illumina iGenomes) sequence was performed using TopHat2 [8]. The merge of lanes and mark of duplicates was performed with gatk (v4.1.2.0. QC and quantification with RNAseQC (v2.3.4) GENCODE v24 annotation, modified according to GTEx collapse gene model. Differential gene expression was conducted with edgeR (v3.26.4.) after gene expression is quantified by counting the number of uniquely mapped reads [9].

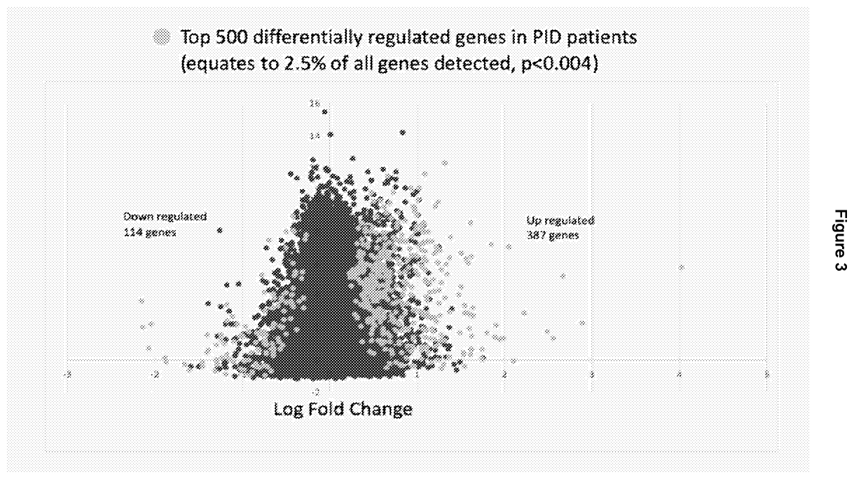

The approach to quantification was to aggregate raw counts of mapped reads using programs such as GTEx or HTSeq-count to obtain gene-level quantitation, and exon level quantification. This and similar alternatives for sequence are outlined by Conesa et al [7]. Exon read counts were retained that have an expression level of at least 2 counts per million reads (CPM) in at least one of the 20 samples. Normalization of RNA profiles adjusting for sequencing depth and other variables was performed using Bioconductor resources [8] and the EdgeR Bioconductor package [9]. FIG. 3 shows a differential gene expression analysis comparing 19,521 genes expressed in blood of PID patients and normal matched controls.

PID is largely a monogenic disease and identification of a known deleterious homozygous mutation (in addition to clinical symptoms) is sufficient to diagnose PID and recommend treatment. Sequence analysis for mutation detection in PID genes was performed by comparison of RNA sequence reads described above to a reference human genome or transcript reference for identification of known deleterious mutations. Paired RNA reads were aligned to genome exons using TOPHAT2 [8] and only those reads that fall within the gene exon boundaries as dictated by UCSC hg19 are used. Each set of alignments from each individual were sorted and indexed using SAMtools [10, 11]. Using the list of known or suspected PID genes and known deleterious mutations from PID discovery projects [12] and that fall within the gene exon boundaries as dictated by UCSC hg19 genome assembly, the SAMtools mpileup function (version 0.1.14) was used to extract informative allele variants in individuals. Additional approaches for variant detection in RNA sequence are becoming available [13].

RNA analysis pipelines can detect homozygous mutations using a set of PID genes and their known mutations and additionally mutations in other suspected genes [12]. Pipelines can also detect heterozygous mutations that contribute to disease phenotypes including those which are dominant mutations, or combinations of different deleterious mutations in the two alleles of the same gene [14]. In addition, RNA analysis pipelines can detect variant SNP that are in close association with causal mutations (and indicate founding mutation haplotypes) [15] that can contribute to diagnosis. In some cases, SNP variation in other parts of the genome may provide information on the likely severity of disease expression in different individuals caused by PID mutations.

Diagnosis of PID in a Subject Using Transcriptomic Best Linear Unbiased Prediction

A prediction equation for PID diagnosis using transcriptomic BLUP was developed from a reference set of transcriptome profiles from normal and PID patients and used to create transcriptomic relationship matrices from which a predictive equation was derived. The reference set of transcriptome profiles was used to create transcriptomic relationship matrices as previously described for microbial molecular signatures [16]. A transcriptome profile is the vector of counts of sequenced reads that align to the collection of human genes (or exon) sequences in the UCSC hg19 genome or reference human transcriptome database. The reads are generated by untargeted sequencing of cDNA derived from RNA. These transcriptome profiles relate to the relative abundance of different mRNA species. The model used assumes a normal distribution, as such the transcriptome profile will be log transformed and standardised. Several transcriptome profiles were combined from an n×m matrix X with elements xᵢⱼ, the log transformed and standardised count for sample i for gene (or exon) j, with n samples and m genes. Genes with <10 reads in total aligning to them were removed from the matrix prior to standardising. These profiles were compared to make a transcriptome relationship matrix (calculated as G=XX′Im). BLUP is used to predict the disease status. A mixed model was fitted to the data: y=1ₙμ+Zg+e. Where y is the vector of disease phenotypes, with one record per sample, 1ₙ is a vector of ones, μ is the overall mean, Z is a design matrix allocating records to samples, and g is a random effect estimate ˜N(0,Gσ²_{g}). The phenotypes y were corrected for other fixed effects such as age and sex prior to analysis. Using ASRemI, σ²_{g} is estimated from the data and the disease status of the samples (ĝ which is a vector of length n) predicted as:

$$[ \begin{matrix}\hat{μ} \\ \hat{g}\end{matrix} ] = {[ \begin{matrix}{1}_{n}^{′} {1}_{n} & {1}_{n}^{′} Z \\ {Z}^{′} {1}_{n} & {Z}^{′} Z + {G}^{- 1} \frac{{σ}_{e}^{2}}{{σ}_{g}^{2}}\end{matrix} ]}^{- 1} [ \begin{matrix}{1}_{n}^{′} y \\ {Z}^{′} y\end{matrix} ]$$

Solving the equations results in an estimate of the mean and an estimate of the residual for each transcriptome profile, such that ĝ has the dimensions n×1. For each transcriptome profile, the predicted disease phenotype was

ĝᵢ+\hat{μ}.

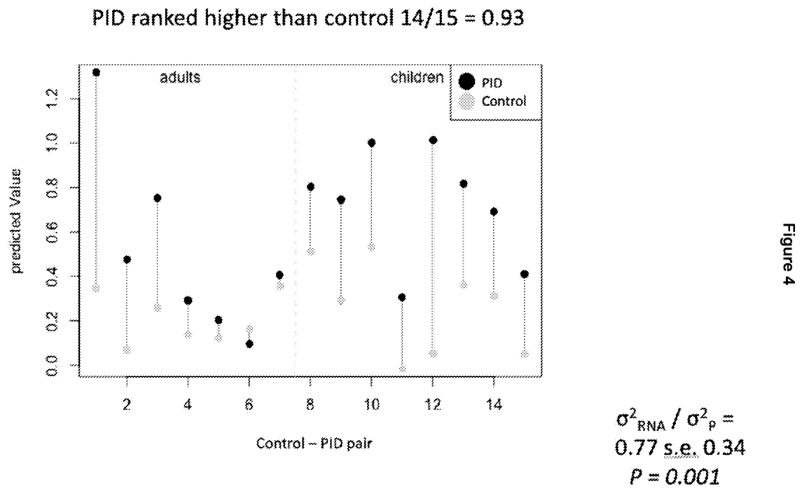

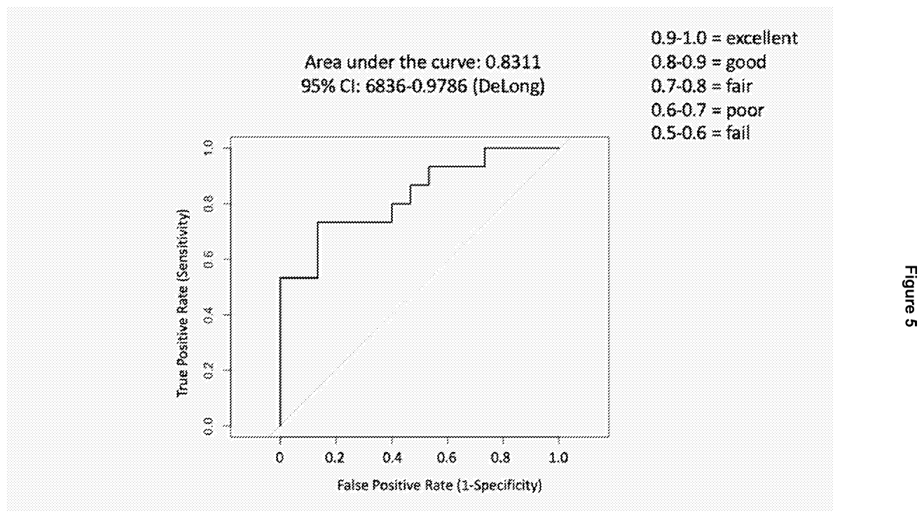

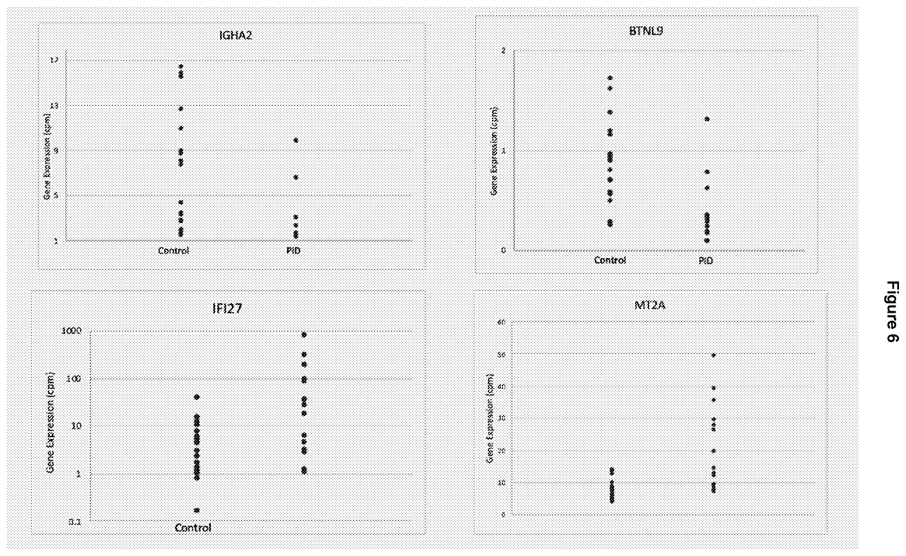

Transcriptome profile prediction for PID was performed in the free R statistical software (version 3.1.2; The R Foundation for Statistical Computing; http://www.r-project.org/) and package rrBLUP [17] was used. A transcriptome relationship matrix was fitted into BLUP and validated using two-fold cross-validation, where PID and non-PID are either training or validation sets, and an alternative procedure called leave-one-out in which one individual is removed sequentially from the dataset to estimate the disease prediction value using the remaining data. Individuals being predicted are always omitted from the training set. FIG. 4 shows the results of application of the predictive model using a leave out one prediction approach. FIG. 5 is an ROC curve demonstrating the utility of the model. Table 1 shows the list of 500 predictive variant genes used in the prediction model. FIG. 6 shows four examples of individual genes that are up or down regulated in PID.

TABLE 1
List of top 500 predictive variant genes in alphabetical order (continued
next page) from both healthy control and PID subjects.

| | | | | | |
|---|---|---|---|---|---|
| ABCG2 | AC002480.2 | AC004817.4 | AC005165.1 | AC005730.1 | AC007952.6 |
| AC010615.2 | AC011379.1 | AC011444.2 | AC017099.1 | AC018755.4 | AC023301.1 |
| AC023355.1 | AC024032.2 | AC024267.1 | AC024940.1 | AC073172.1 | AC087203.2 |
| AC087481.1 | AC092490.1 | AC092802.1 | AC092821.1 | AC093909.6 | AC099489.3 |
| AC099521.3 | AC103810.5 | AC104090.1 | AC104389.2 | AC104809.2 | AC109326.1 |
| AC111000.4 | AC123912.4 | AC124312.3 | AC126544.1 | AC130366.1 | AC133919.3 |
| AC136475.5 | AC243829.1 | AC253572.1 | ACHE | ACKR1 | ADAM29 |
| ADARB2 | ADRA2A | ADRB1 | ADTRP | AGGF1P1 | AHSP |
| AJAP1 | AL008636.1 | AL008707.1 | AL031432.2 | AL031593.1 | AL121835.2 |
| AL139220.2 | AL139276.1 | AL157895.1 | AL161781.2 | AL353597.3 | AL353616.1 |
| AL353729.2 | AL356585.1 | AL391097.1 | AL590399.5 | AL592158.1 | AL645929.1 |
| AL773545.3 | ALAS2 | ALOX15 | ALOX15B | ALPK2 | ALPL |
| ANKRD20A11P | ANKRD20A4P | ANKRD22 | AOC1 | AOC3 | AP000350.2 |
| AP000350.6 | APOBEC3B | APOL4 | ARHGAP8 | ATOH8 | ATP1A4 |
| ATP1B2 | B4GALNT3 | B4GALNT4 | BATF2 | BCAM | BCL2L1 |
| BEND3P1 | BMP3 | BTNL9 | C14orf132 | C17orf99 | C19orf33 |
| C1QB | C4BPA | CA1 | CA3-AS1 | CACNG6 | CAV1 |
| CCDC144A | CCL3L3 | CCNA1 | CD177 | CD19 | CD22 |
| CDH2 | CEACAMP3 | CEROX1 | CFH | CHL1 | CHST8 |
| CICP27 | CLC | CLEC4F | CLRN1 | CMBL | CNR1 |
| CNTNAP2 | CNTNAP3 | COL19A1 | CPA3 | CPSF1P1 | CRYM |
| CSMD1 | CTNNAL1 | CTSE | CTSG | CTTNBP2 | CTXN2 |
| CXCL10 | CXCL8 | CYP4F29P | DAAM2 | DAAM2-AS1 | DAB1 |
| DACT1 | DDX11L10 | DEFA1 | DEFA3 | DEFA4 | DIPK2B |
| DLGAP1 | DMC1 | DSC1 | DSP | DUX4L9 | EDA |
| EGR1 | EIF3CL | ELAPOR1 | EPB42 | ETV7 | FAM106A |
| FAM106A | FAM153CP | FAM157A | FAM210B | FAT1 | FCRL5 |
| FCRLA | FKBP1B | FOLR3 | FREM3 | GAPDHP14 | GATA2 |
| GBP1P1 | GIMAP3P | GLDC | GPM6A | GPX1P1 | GRIK4 |
| GSTM1 | GSTM3 | GTF2H2B | GYPB | H2BP2 | HBG2 |
| HBM | HBQ1 | HDC | HEPACAM2 | HEPH | HERC2P10 |
| HLA-DQA2 | HLA-DQB1 | HLA-DQB1- | HLA-DQB2 | HLA-DRB5 | HLA-G |
| | | AS1 | | | |
| ICOSLG | IFI27 | IFI44 | IFI44L | IFIT1 | IFIT1B |
| IGF2 | IGFBP2 | IGHA1 | IGHA2 | IGHD | IGHG1 |
| IGHG2 | IGHG3 | IGHG4 | IGHM | IGHV1-2 | IGHV1-24 |
| IGHV1-3 | IGHV1-69D | IGHV2-26 | IGHV2-5 | IGHV2-70 | IGHV3-13 |
| IGHV3-15 | IGHV3-21 | IGHV3-23 | IGHV3-33 | IGHV3-48 | IGHV3-49 |
| IGHV3-53 | IGHV3-7 | IGHV3-74 | IGHV4-39 | IGHV4-4 | IGHV4-59 |
| IGHV5-10-1 | IGKC | IGKV1-12 | IGKV1-16 | IGKV1-17 | IGKV1-27 |
| IGKV1-33 | IGKV1-39 | IGKV1-5 | IGKV1-6 | IGKV1-9 | IGKV1D-33 |
| IGKV1D-39 | IGKV1D-8 | IGKV2-24 | IGKV2-30 | IGKV2D-28 | IGKV2D-29 |
| IGKV3-11 | IGKV3-15 | IGKV3-20 | IGKV4-1 | IGLC1 | IGLC2 |
| IGLC3 | IGLC7 | IGLV1-40 | IGLV1-44 | IGLV1-47 | IGLV2-8 |
| IGLV3-1 | IGLV3-19 | IGLV3-21 | IGLV3-25 | IGLV5-45 | IGLV6-57 |
| IGLV7-43 | IGLV7-46 | IGLV8-61 | IL1RL1 | IL5RA | INTS4P1 |
| IRF6 | ISG15 | ISM1 | ITGA2B | ITLN1 | JCHAIN |
| KANK2 | KAZN | KCNG1 | KCNH2 | KIAA0319 | KIR2DS4 |
| KIR3DL1 | KIR3DL2 | KLHL14 | KRT1 | KRT72 | KRT73 |
| KRT73-AS1 | LAIR2 | LARGE1 | LEP | LGSN | LINC00189 |
| LINC00570 | LINC00683 | LINC00824 | LINC01291 | LINC01293 | LINC01876 |
| LINC02073 | LINC02141 | LINC02193 | LINC02288 | LINC02289 | LINC02397 |
| LINC02458 | LINC02470 | LINC02596 | LMOD1 | LPAR3 | LPL |
| LRP1B | LRRC2 | LTF | LY6G6E | LYPD2 | MACROD2 |
| MAGI2-AS3 | MAOA | MAP7D2 | MARCO | MDGA1 | MEG3 |
| MFSD2B | MS4A2 | MT1L | MTDHP3 | MTND3P9 | MYL4 |
| MYO3B | MYOM2 | MZB1 | NAIPP3 | NBPF13P | NEBL |
| NEFL | NETO1 | NEXMIF | NF1P8 | NKX3-1 | NOG |
| NRCAM | NRXN3 | NSFP1 | NT5M | NTN4 | OCLNP1 |
| OLFM4 | OR2AK2 | OR2L9P | OR2T8 | OR2W3 | ORM1 |
| OSBP2 | OTOF | OVCH1 | OVCH1-AS1 | PAGE2B | PAQR9 |
| PAX5 | PAX8-AS1 | PAX8-AS1 | PCDHGA5 | PCDHGB2 | PDZK1IP1 |
| PGM5 | PHF24 | PI3 | PLSCR4 | PLVAP | PPP4R4 |
| PRKY | PRSS33 | PSMA6P1 | PTGES | PTGFR | PTPN20 |
| PWP2 | PXDN | RAP1GAP | RHD | RN7SL3 | RNASE3 |
| RNF182 | RNY1 | RNY3 | ROBO1 | RP11- | RP11- |
| | | | | 706O15.3 | 706O15.5 |
| RPL13P12 | RPL3L | RPL9P33 | RPSAP47 | RSAD2 | RUNDC3A |
| S100B | S100P | SAXO2 | SCARNA5 | SDK2 | SEC14L3 |
| SEC14L5 | SELENBP1 | SERPINB10 | SERPING1 | SGCD | SGIP1 |
| SIGLEC1 | SIGLEC12 | SIGLEC14 | SIGLEC8 | SLC12A1 | SLC2A14 |
| SLC2A4 | SLC38A11 | SLC44A4 | SLC44A5 | SLC4A1 | SLC5A4-AS1 |
| SLC6A19 | SLC6A8 | SLC6A9 | SMARCA1 | SMIM1 | SMIM24 |
| SMN2 | SMPD4P1 | SNORA23 | SNORA47 | SNORA49 | SNORA53 |
| SNORA68 | SNORA80B | SNORD3A | SNORD3B-1 | SNORD3C | SNTG2 |
| SNX18P13 | SNX18P9 | SORCS3 | SOX5 | SPP1 | SPTB |
| STOX1 | SYCP2L | TACSTD2 | TAS2R41 | TAS2R43 | TAS2R60 |
| TAS2R62P | TAS2R64P | TBC1D27P | TENM4 | TENT5C | TGM3 |
| THEGL | TMCC2 | TMEM158 | TMEM176A | TMEM176B | TMTC1 |
| TNFRSF13B | TNFRSF17 | TNR | TNS1 | TRBV30 | TRDV2 |
| TREML5P | TSIX | TSPAN7 | TSPEAR | TSPEAR-AS1 | TSPEAR-AS2 |
| TTC4P1 | TUBB2A | TUBB2B | TUBBP5 | U2AF1 | UBBP1 |
| UGT2B11 | USP32P1 | USP32P2 | VMO1 | VWCE | VWDE |
| WDR63 | WNT7A | XIST | XK | XKR3 | Y_RNA |
| ZFP57 | ZMAT4 | ZNF208 | ZNF215 | ZNF462 | ZNF727 |
| ZNF860 | ZNF890P | | | | |

Increasing the transcriptome sample reference numbers from affected and unaffected individuals facilitates additional training for the transcriptomic BLUP and iteratively increases accuracy of prediction and diagnosis.

TABLE 2
List of top 500 differentially regulated genes in alphabetical order (continued
next page) in subjects with PID compared to healthy controls.

| | | | | | |
|---|---|---|---|---|---|
| ABCC13 | AC004987.9 | AC007365.3 | AC023590.1 | AC026271.5 | AC092580.4 |
| ACER3 | ACHE | ACKR1 | ACP1 | ACVR1C | ADAM9 |
| ADIPOR1 | AHSP | AKAP6 | ALAS2 | AMPD2 | ANKRD22 |
| ANP32B | ANXA2 | ANXA2P2 | AP2M1 | AP2S1 | APOL3 |
| APOL4 | APOLD1 | APOPT1 | ASCL2 | ASNA1 | ATP5B |
| ATP5E | ATP5J2 | ATP6V0C | AURKA | BAMBI | BCAM |
| BCAS4 | BEND5 | BISPR | BLVRA | BSCL2 | BSG |
| BST2 | BTF3 | BTNL9 | C16orf74 | C17orf99 | C18orf8 |
| C2 | CA1 | CALCOCO2 | CAPG | CAPNS1 | CARD16 |
| CARM1 | CASP1 | CASP7 | CBX7 | CCL5 | CCRL2 |
| CD177 | CD200 | CD33 | CD36 | CD3EAP | CD68 |
| CD8A | CDAN1 | CDC34 | CDCA7 | CDKL1 | CEBPA |
| CERK | CETP | CFAP45 | CHMP4B | CHPT1 | CISD2 |
| CITED2 | CLDN5 | CLEC11A | CLEC1B | CLEC6A | CLEC9A |
| CMPK2 | CNN3 | CNPPD1 | CNTLN | COA6 | COCH |
| COL27A1 | COL4A3 | COL6A1 | COMT | COX5A | COX6B1 |
| CPNE8 | CREG1 | CROCCP2 | CRYM | CSTB | CTB- |
| | | | | | 193M12.5 |
| CTD- | CTD- | CTD- | CTD- | CTD- | CTNNAL1 |
| 2002H8.2 | 2319I12.10 | 2540L5.6 | 2619J13.14 | 3252C9.4 | |
| CTSB | CXCL10 | CYB5A | CYBB | CYTH2 | DAAM2 |
| DCP1B | DDIAS | DDX60 | DESI1 | DHX58 | DLGAP1 |
| DLGAP5 | DPCD | DPM2 | DRAP1 | DTL | DYNLL1 |
| DYNLRB1 | E2F1 | EFCAB2 | EIF2AK2 | EIF4EBP1 | EIF5AL1 |
| EMC3 | EMID1 | ENC1 | EOMES | EPB41L3 | EPOR |
| EPSTI1 | ETV7 | FADS1 | FAHD1 | FAM104A | FAM104B |
| FAM132B | FAM177B | FAM210B | FAT4 | FBLN2 | FBXO6 |
| FCER1G | FCGR1C | FGFR1OP | FIS1 | FKBP1B | FKBP8 |
| FRMD3 | FTL | FTLP3 | FUCA1 | FUNDC2 | GABARAP |
| GABARAPL2 | GALM | GBP1 | GBP1P1 | GBP5 | GCNT1P3 |
| GLRX5 | GNAS | GNG7 | GOLGA8R | GP9 | GPD2 |
| GPR137B | GPR146 | GPR150 | GPR61 | GPR84 | GPS2 |
| GPX1 | GPX4 | GRIK4 | GSPT1 | GSTK1 | GYPC |
| HBA1 | HBA2 | HBG2 | HBM | HCST | HDGF |
| HDX | HERC5 | HIST1H2BN | HP | HPS1 | HSPB1 |
| IDH1 | IDH2 | IFI27 | IFI27L2 | IFI35 | IFI44 |
| IFI44L | IFI6 | IFIT1 | IFIT1B | IFIT3 | IFITM1 |
| IGF2 | IGHA2 | IGHG2 | IGHG4 | IGHV2-26 | IGKV1-8 |
| IGKV1D-8 | IGLV5-45 | IGSF9 | IL15 | IL15RA | IL1RAP |
| INTS12 | ISG15 | ITSN1 | JAK2 | JHDM1D-AS1 | JUND |
| KCNH2 | KCNH8 | KCNK5 | KEL | KRT1 | KRT72 |
| KRT73 | KRT73-AS1 | LAP3 | LDLR | LGALS3 | LHFPL2 |
| LINC00534 | LINGO2 | LPXN | LRRC8A | LSM12P1 | LY6E |
| LYL1 | MAF1 | MARCO | MASTL | MBNL1-AS1 | MCOLN1 |
| MCOLN2 | MED16 | MEG3 | METTL9 | MFSD2B | MGST1 |
| MIIP | MPP1 | MRPS17P5 | MSC-AS1 | MSMO1 | MT1E |
| MT1F | MT1L | MT2A | MTCO2P11 | MTCO3P11 | MTHFD1 |
| MX1 | MYBL2 | MYOM2 | NAPA | NDUFA5P11 | NDUFAF3 |
| NDUFB8P2 | NDUFS7 | NDUFV3 | NEIL1 | NEIL3 | NRG1 |
| NRIR | NT5C3A | NT5E | NT5M | NTAN1 | NUCB1 |
| NUDT14 | NUDT19P5 | OAS1 | OAS2 | OAS3 | OASL |
| OGN | OLA1P1 | OST4 | PA2G4 | PAIP2B | PAQR9 |
| PARP12 | PARP14 | PARP9 | PARPBP | PBXIP1 | PCBP3 |
| PCED1A | PCTP | PDE3B | PDE6A | PDK4 | PFDN2 |
| PHACTR1 | PHF11 | PHF24 | PIGC | PLA2G7 | PLEK2 |
| PLK3 | PLOD2 | PNP | PQLC1 | PRDX2 | PRR5 |
| PSEN2 | PSMB2 | PSME1 | PSME2 | PSTPIP2 | PTTG1 |
| RAB39A | RAP1GAP | RASGRP2 | RASSF6 | RBM11 | RBX1 |
| RETN | REXO2 | RFX2 | RGS10 | RHOU | RILP |
| RN7SKP296 | RN7SL128P | RN7SL4P | RNA5SP202 | RNF187 | RNY1 |
| RP1- | RP1- | RP11- | RP11- | RP11- | RP11- |
| 167A14.2 | 257A7.4 | 102N12.3 | 103B5.4 | 1193F23.1 | 12A2.1 |
| RP11- | RP11- | RP11- | RP11- | RP11- | RP11- |
| 153M7.3 | 158I9.5 | 162A23.5 | 20D14.6 | 288L9.4 | 305L7.1 |
| RP11- | RP11- | RP11- | RP11- | RP11- | RP11- |
| 403B2.7 | 422P24.12 | 466G12.3 | 474P2.4 | 500G10.5 | 609D21.3 |
| RP11- | RP11- | RP11- | RP11- | RP11- | RP11- |
| 61I13.3 | 676J12.9 | 68I3.5 | 70C1.1 | 713P17.3 | 77H9.8 |
| RP11- | RP11- | RP11- | RP11- | RP11- | RP4- |
| 798G7.6 | 7F17.3 | 81H14.2 | 886P16.10 | 96K19.4 | 641G12.3 |
| RP5- | RP5- | RPA4 | RPS27P16 | RPSAP6 | RSAD2 |
| 1028K7.2 | 998N21.4 | | | | |
| RSRC1 | RSU1 | RTP4 | RUFY4 | RUNDC3A | S100A12 |
| S100A4 | SAMD9L | SAP30 | SCD | SDC2 | SDSL |
| SELENBP1 | SELK | SERF2 | SERPINB9 | SERPING1 | SERTAD2 |
| SESTD1 | SGIP1 | SHARPIN | SHC3 | SHTN1 | SIAH2 |
| SIGLEC1 | SKA3 | SLC1A3 | SLC30A4 | SLC6A19 | SLC9A7 |
| SLFN11 | SLFN12 | SMIM1 | SMIM24 | SNTB1 | SNX15 |
| SNX3 | SP100 | SPATS2L | SPHK1 | SPOCD1 | SPRY1 |
| SPSB2 | SQLE | ST13P6 | STAT1 | STAT2 | STIL |
| STK11 | STOM | STYK1 | SVBP | SWAP70 | SYBU |
| TAGLN2 | TCN2 | TCTN1 | TDRD6 | TESC | TFEC |
| TFR2 | THOC7 | THRA | TICAM2 | TLR7 | TM7SF2 |
| TMEM158 | TMSB4XP8 | TNFSF13 | TNNT1 | TOMM6 | TOR1A |
| TPGS1 | TPGS2 | TPM1 | TRBV7-6 | TRBV7-9 | TRBV9 |
| TRIM22 | TRNP1 | TSC22D3 | TSPAN17 | TSPO2 | TSTA3 |
| TTC9 | TUBBP5 | TXNL4A | TYMP | UBA52 | UBAP2 |
| UBB | UBBP4 | UBE2F | UBE2L6 | UBL7 | UGT8 |
| USP18 | UST-AS1 | VCAN | VRK2 | WARS | WASF4P |
| XAF1 | XKRX | XXyac- | YARS | YBX1P10 | ZBP1 |
| | | YR38GF2.1 | | | |
| ZDHHC23 | ZDHHC4P1 | ZMAT2 | ZNF662 | ZNF677 | ZNF711 |
| ZNF772 | ZNRF1 | | | | |

Metagenome Profile Determination

Untargeted massively parallel sequencing of ribosomal or microbial DNA was performed to generate reference PID metagenome profiles.

i) Sample Collection and DNA Extraction

For microbiome profile acquisition, DNA was extracted from buccal swabs and hair follicles using DNA extraction kits as described below.

Buccal Swap Sample Collection:
1. Teeth are brushed sometime within the 4 hours before sampling with eating avoided (following teeth brushing) prior to sampling.
2. The cotton swab or nylon brush was used to push and swipe the buccal mucosa.
3. Cotton was stripped from the swab with sterile tweezers and placed into a tube containing lysis buffer or the head of the nylon brush was placed directly into a tube containing lysis buffer.

Extracting DNA from Buccal Swabs

Materials:
1. QIAamp DNA Mini Kit (Qiagen, Cat. Cat No./ID: 51304, Cat No./ID: 51306)
2. RNase A solution (R6148-25 ml, Sigma)
3. Preparation of lysis buffer: 20 mg lysozyme in the solution of 25 mM Tris.HCl, pH8.0; 2.5 mM EDTA, pH8.0 and 1% Triton X-100

Protocol:
Place buccal swab (cotton) in a 1.5 mL or 2 mL tube.
Add 400 μl lysis buffer (20 mg/ml lysozyme in the solution of 25 mM Tris.HCl, pH 8.0; 2.5 mM EDTA, pH8.0 and 1% Triton X-100). Mixed by pushing cotton several time and pipetting.
Incubate at 37° C. for 60 min.
Add 40 μl proteinase K (20 mg/ml) and 400 μl Buffer AL. Mix thoroughly by vortex for 10 seconds. (Note: do not mix proteinase K directly to Buffer AL.) Briefly centrifuge the tube to remove drops from inside the lid.
Incubate at 55° C. for 60 min. Vortex occasionally during incubation to disperse the sample.
Incubate further 15 min at 80° C. to inactivate the proteinase K.
Remove the solution to a new tube. Push tightly cotton using a pipette tip and remove the solution as possible.
Add RNase A solution (R6148-25 ml, Sigma) 8 μl, 37° C. for 60 min.
Add 450 μl ethanol (96-100%) to the sample and mix by pulse-vortexing for 15 s. Briefly centrifuge the tube to remove drops from inside the lid.
Apply the mixture (including all the precipitate, need to divide mixture into 2 portions) to the Mini spin column. Close the cap and centrifuge at maximum speed for 1 min.
Add 500 μl Buffer AW1 to the column. Close the cap and centrifuge at maximum speed for 1 min and discard the filtrate.
Add 500 μl Buffer AW2 to the column. Close the cap and centrifuge at maximum speed for 1 min and discard the filtrate.
Transfer the column to new 2 ml collection tube and centrifuge at maximum speed for 2 min.
Put column in a 1.5 ml tube and add 50-100 μl Buffer EB (Qiagen) or 10 mM Tris-HCl, pH 8.5, depending on gDNA concentration required. Incubate at room temperature for 1-3 min and then centrifuge at maximum speed for 2 min.

For skin sampling, skin preparation instructions include avoiding bathing and avoiding emollients or antimicrobial soaps or shampoos for 12 hours prior to all sampling. Sampling sites include the retroauricular crease, cubital fossa or volar forearm. From a 4 cm² area, bacterial swabs (via Epicentre swabs) and scrapes (via sterile disposable surgical blade) are obtained and incubated in an enzymatic lysis buffer and lysozyme as described above for buccal swab samples.

Amplification of Microbial DNA from Buccal Swabs

PCR 16S PCR

Primers used: 341F/806R primers which cover 16S V4 region. The primer sites are targeted by the “forward” primer 341F and the “reverse” primer 806R. In addition, bar coding primers for Illumina MySeq sequencing are included (shaded below).

Illumina Multiplexing Read1 Sequencing Primer was
added (806R Ad)
(SEQ ID NO: 1)

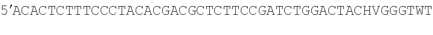

CTAAT3′
Illumina Multiplexing Read2 Sequencing Primer was
added (341F Ad)
(SEQ ID NO: 2)

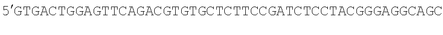

AG 3'

TABLE 3
PCR reaction set up: 50 μl reaction

| Component | Final conc. | Vol (μl) |
|---|---|---|
| 5 X Phusion HF buffer | 1 X | 10 |
| 10 mM dNTP | 0.2 mM each | 1 |
| Primer mix (10 μM each) | 0.1 μM each | 0.5 |
| DMSO | 5% | 2.5 |
| Phusion DNA polymerase | 0.02 U/μl | 0.5 |
| Water | | 33.5 |
| gDNA Template | | 2 |
| Total | | 50 |

TABLE 4
Thermal cycler conditions

| | | | | | | | | | | |
|---|---|---|---|---|---|---|---|---|---|---|
| Temp. | 98° | C. | 98° | C. | 60° | C. | 72° | C. | 72° | C. |
| Time | 30 | sec | 10 | sec | 15 | sec | 15 | sec | 1 | min |

| | | | |
|---|---|---|---|
| Cycle | 1 | 30 | 1 |

Sequencing of Microbial DNA from Buccal Swabs

Standard Illumina Protocols Used for Sequencing MiSeq.

ii) Targeted and Untargeted Massively Parallel Sequencing

Library preparation for sequencing was performed using indexing protocol using IIlumina barcoding primers as described by the manufacturer. The indexes are a short third read of the sequencing run. Briefly, DNA is sheared to 300 bp, adapters are added by ligation, and then indexes added using PCR. The libraries are then quantified and pooled. Paired-end sequencing of genomic DNA was performed on a HiSeg2000™ sequencer. Sequence reads were trimmed so that the average Phred quality score for each read will be above 20. If the read length is below 50 after trimming, the read was discarded.

iii) Metagenome Profile Analysis

Diagnosis of PID in a Subject Using Metagenomic Best Linear Unbiased Prediction

The reference set of metagenome profiles generated were used to create metagenomic relationship matrices essentially as previously described [16]. A metagenome profile is the vector of counts of sequenced reads that align to a collection of 16S rRNA sequences or other available or generated reference sequence sets (here referred to as contigs) in a database. The reads were generated by untargeted sequencing of microbial DNA, or by sequencing 16S ribosomal sequences amplified by PCR from microbial DNA. These metagenome profiles relate to the relative abundance of different microbial species. The model used assumes a normal distribution, as such the metagenome profile will be log transformed and standardised.

Several metagenomic profiles were combined from an n×m matrix X with elements xᵢⱼ, the log transformed and standardised count for sample i for contig j, with n samples and m contigs. Contigs with <10 reads in total aligning to them will be removed from the matrix prior to standardising. These profiles are compared to make a microbiome relationship matrix (calculated as G=XX′/m). Best linear unbiased prediction was used to predict the phenotype. A mixed model was fitted to the data: y=1ₙμ+Zg+e. Where y is the vector of clinical phenotype, with one record per sample, 1ₙ is a vector of ones, μ is the overall mean, Z is a design matrix allocating records to samples, and g is a random effect estimate ˜N(0,Gσ²_{g}). Using ASRemI, σ²_{g} was estimated from the data and the phenotypes of the samples (ĝ which is a vector of length n) predicted as:

$$[ \begin{matrix}\hat{μ} \\ \hat{g}\end{matrix} ] = {[ \begin{matrix}{1}_{n}^{′} {1}_{n} & {1}_{n}^{′} Z \\ {Z}^{′} {1}_{n} & {Z}^{′} Z + {G}^{- 1} \frac{{σ}_{e}^{2}}{{σ}_{g}^{2}}\end{matrix} ]}^{- 1} [ \begin{matrix}{1}_{n}^{′} y \\ {Z}^{′} y\end{matrix} ]$$

Solving the equations results in an estimate of the mean and an estimate of the residual for each metagenome profile, such that ĝ has the dimensions n×1. For each metagenome profile, the predicted phenotype is ĝᵢ+\hat{μ}.

Microbiome profile prediction for PID was performed in the free R statistical software (version 3.1.2; The R Foundation for Statistical Computing; http://www.r-project.org/) and package rrBLUP [17] were used. A metagenomics relationship matrix was fitted into best linear regression model (BLUP) and validated using two-fold cross-validation, where PID and non-PID are either training or validation sets, and an alternative procedure called leave-one-out in which one individual is removed sequentially from the dataset to estimate the disease prediction value using the remaining data. Individuals being predicted were always omitted from the training set.

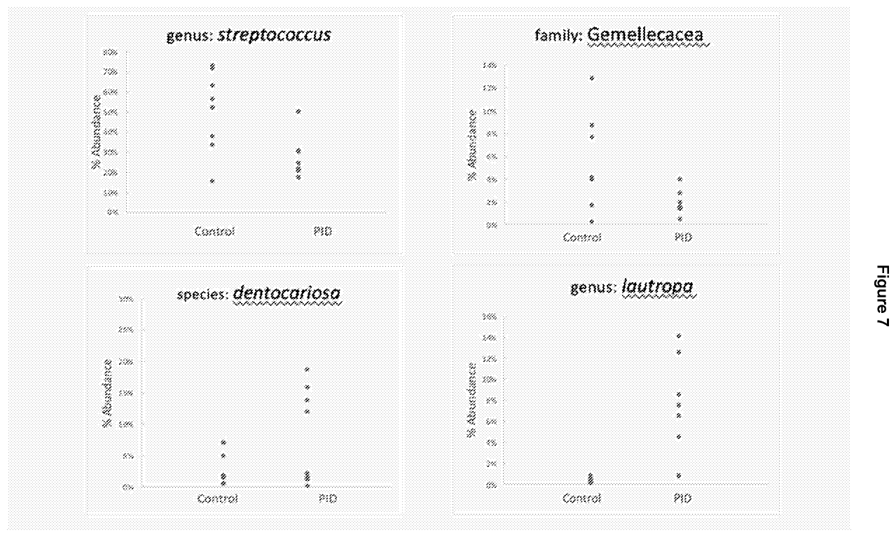

Updating the microbiome sample reference numbers from affected and unaffected individuals for training the metagenomics BLUP (or BayesR) as described above iteratively increases prediction accuracy. FIG. 7 shows an analysis demonstrating examples of significant differences in specific microbes between PID patients and age and sex matched controls.

Diagnosis of PID in a Subject by Combined RNA and Metagenomic Best Linear Unbiased Prediction

Integrative (transcriptomics and metagenomics) prediction was performed in R statistical software. Twenty positive and 20 negative diagnoses for PID and blood transcriptomic profile and metagenomic profile were fitted into a linear regression model.

An extended relationship matrix was developed that combines the Z matrix described above for RNA transcript abundances with the metagenomic Z¹ relationship matrix as follows:

y=1ₙμ+Zg+Z¹g¹+e

The coefficients in the output were multiplied with blood transcriptomic profile and metagenomic profile respectively to calculate the integrative predicted PID disease phenotype. Accuracy of prediction was assessed by Pearson's correlation, cry, that is, the correlation between the measured values with predicted values.

The results demonstrated that: firstly, transcriptome profiles were able to predict PID in these circumstances; secondly, integrating transcriptomic with metagenomics information can increase prediction accuracy. Updating the transcriptome and microbiome sample reference for training with affected and unaffected individuals will increase prediction accuracy.

Diagnosis of PID in a Subject by Gene Sequence Based Prediction

PID is largely a monogenic disease and identification of a known homozygous mutation (in addition to clinical symptoms) is enough to diagnose PID and recommend treatment, and this information can be derived from RNA sequence as described above. In addition, where expression of a PID gene normally expressed in blood is not detected in blood this also indicates a serious regulatory defect or destabilising mutation, and RNAseq can reveal these serious defects in expression directly, even in the absence of causative mutation confirmation to allow a diagnosis.

Other genomic variants in mRNA such as SNP variants that are in linkage with defective immune genes in the population may be useful for prediction. A known mutation in mRNA may be inferred or imputed from linkage to a co-occurring haplotype marker in the mRNA expressed from the same gene, or nearby gene on the chromosome. This genomic information can be obtained from genomic sequence or RNA sequence and used to inform diagnosis alone or in combination with transcriptomic BLUP or transcriptomic BayesR.

Manifestation of the same PID disease mutation varies between individuals [18] and various genome variants may influence severity of the disease and measuring this contributing or protective variation may be useful in assisting with predicting less severe or later onset cases of PID where the disease is more weakly expressed. Once enough patient samples are obtained, this type of variation in the genome detected through RNA sequence (and/or whole genome or exome sequence) will become more useful in assisting predicting disease severity. These may also assist in better diagnosing autoimmune manifestations of PID [19] or PID cases that include autoimmune symptoms.

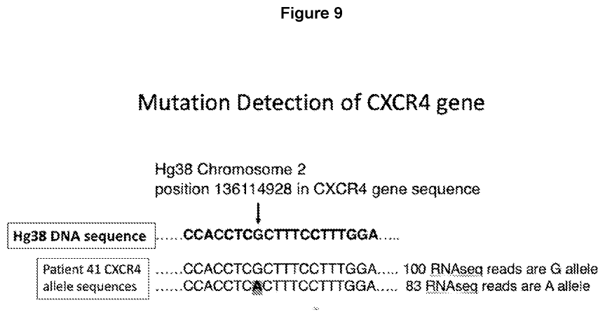

The patients included in the study had known genetic mutations, identified through DNA sequencing, which are causative of their disease. In order to evaluate if the PID genes are transcribed at sufficient levels in blood such that gene mutations are also able to be identified at the mRNA level in RNAseq data, levels of PID gene transcript expression were determined in a number of individuals. By examining the number of sequence reads covering PID genes it is possible to determine the possibility for detecting mutations in RNA. FIG. 7 demonstrates detection of sufficient gene expression of several PID genes in PID patients. Using the CXCR4 gene as an example of successful mutation detection, using RNAseq, the dominant missense gene mutation causative of disease was identified (FIG. 9). In whole blood RNAseq data obtained from PID patient 41, a total of 183 mRNA sequence reads covered a region in the CXCR4 mRNA sequence where a mutated allele (in the position marked by the arrow) was observed (83 copies) and normal allele variant sequences (100 copies) were observed and determined. The A base variant at this location in Chromosome 2 is a missense mutation creating a stop codon in the coding sequence (arg to STOP) of CXCR4 known to cause PID (FIG. 9).

Diagnosis of PID in a Subject by Different Linear Mixed Model Approaches

Alternative linear mixed model approaches to BLUP such as BayesR applied to genomic prediction based on across genome sequence variation as described by Kemper et al [20] can also be applied to transcriptomic and/or metagenomics data in the same way as has been described above using BLUP, with the X matrix describing individuals by normalised read counts per contig. The BayesR method assumes that the true effects of gene expression are derived from a series of normal distributions, the first with zero variance, up to one with moderate to large variance. The advantage of BayesR over BLUP is that the effects of individual genes are not compressed as hard towards the mean as in BLUP. BayesR approaches can also be extended as described by MacLeod et al [21] to include known biological information (BayesRC) such as immune system regulatory function.

Diagnosis of PID in a Subject by Machine Learning Approaches

Alternative approaches to linear mixed models may also be applied to transcriptomic and/or metagenomic data in a predictive way similar to what has been described above using BLUP to enable classification and prediction of PID. Machine learning, support vector machines, and neural networks can provide an alternate approach to linear mixed models for using transcriptomic and/or metagenomic data from patients and normal controls as input for patient classification, and subsequently predictive model training. A similar approach has been used for classifying cancer patients into high or low risk groups and for the development of predictive models to assist prognosis [22], and using tumour RNAseq data as input for this purpose is being investigated [23].

The linear mixed models may be coupled with a descriptive report from the subject that would be useful for an informed clinician to assist with assessment of immune system dysregulation, cells and pathways effected, disease status and perhaps preferred treatment.

To do this genes significantly differentially expressed in a given PID patient (for example, 20 or 50 or even 100 DE genes) can be identified, and this DE gene set subject to a pathway over-representation analysis (or Gene Set Enrichment Analysis), such as or similar to DAVID or Reactome programs and from them a report generated on the pathway and cellular functions that are affected in the subject.

A further qualitative report to supplement diagnosis could be to provide a clustering report of how the subjects transcriptome compares to other patients in the database. This can be based on the transcriptomic relationship matrix or other analysis of the differential gene expression from that patient. It would be understood that patients with mutations in the same gene cluster closer together based on their transcriptome. Once a larger database of patients is available, this clustering may assist in categorisation of newly diagnosed patients into PID disease subtype based on transcriptome (to complement and mutation detection similarities found).

### REFERENCES

1. Salem S, Langlais D, Lefebvre F, Bourque G, Bigley V, Haniffa M, Casanova J L, Burk D, Berghuis A, Butler K M et al: Functional characterization of the human dendritic cell immunodeficiency associated with the IRF8(K108E) mutation. Blood 2014, 124(12):1894-1904.
2. Naik S, Bouladoux N, Wilhelm C, Molloy M J, Salcedo R, Kastenmuller W, Deming C, Quinones M, Koo L, Conlan S et al: Compartmentalized control of skin immunity by resident commensals. Science 2012, 337(6098):1115-1119.
3. Oh J, Freeman A F, Park M, Sokolic R, Candotti F, Holland S M, Segre J A, Kong H H: The altered landscape of the human skin microbiome in patients with primary immunodeficiencies. Genome research 2013, 23(12):2103-2114.
4. Gallo V, Dotta L, Giardino G, Cirillo E, Lougaris V, D′Assante R, Prandini A, Consolini R, Farrow E G, Thiffault I et al: Diagnostics of Primary Immunodeficiencies through Next-Generation Sequencing. Frontiers in immunology 2016, 7:466.
5. Erbe M, Hayes B J, Matukumalli L K, Goswami S, Bowman P J, Reich C M, Mason B A, Goddard M E: Improving accuracy of genomic predictions within and between dairy cattle breeds with imputed high-density single nucleotide polymorphism panels. Journal of dairy science 2012, 95(7):4114-4129.
6. Zhang W, Yu Y, Hertwig F, Thierry-Mieg J, Thierry-Mieg D, Wang J, Furlanello C, Devanarayan V, Cheng J, Deng Y et al: Comparison of RNA-seq and microarray-based models for clinical endpoint prediction. Genome biology 2015, 16:133.
7. Conesa A, Madrigal P, Tarazona S, Gomez-Cabrero D, Cervera A, McPherson A, Szczesniak M W, Gaffney D J, Elo L L, Zhang X et al: A survey of best practices for RNA-seq data analysis. Genome biology 2016, 17:13.
8. Huber W, Carey V J, Gentleman R, Anders S, Carlson M, Carvalho B S, Bravo H C, Davis S, Gatto L, Girke T et al: Orchestrating high-throughput genomic analysis with Bioconductor. Nature methods 2015, 12(2):115-121.
9. Robinson M D, McCarthy D J, Smyth G K: edgeR: a Bioconductor package for differential expression analysis of digital gene expression data. Bioinformatics 2010, 26(1):139-140.
10. Etherington G J, Ramirez-Gonzalez R H, MacLean D: bio-samtools 2: a package for analysis and visualization of sequence and alignment data with SAMtools in Ruby. Bioinformatics 2015, 31(15):2565-2567.
11. Li H, Handsaker B, Wysoker A, Fennell T, Ruan J, Homer N, Marth G, Abecasis G, Durbin R: The Sequence Alignment/Map format and SAMtools. Bioinformatics 2009, 25(16):2078-2079.
12. Itan Y, Casanova J L: Novel primary immunodeficiency candidate genes predicted by the human gene connectome. Frontiers in immunology 2015, 6:142.
13. Sheng Q, Zhao S, Li C I, Shyr Y, Guo Y: Practicability of detecting somatic point mutation from RNA high throughput sequencing data. Genomics 2016, 107(5):163-169.
14. Lionakis M S: Genetic Susceptibility to Fungal Infections in Humans. Current fungal infection reports 2012, 6(1):11-22.
15. Hsu A P, Sampaio E P, Khan J, Calvo K R, Lemieux J E, Patel S Y, Frucht D M, Vinh D C, Auth R D, Freeman A F et al: Mutations in GATA2 are associated with the autosomal dominant and sporadic monocytopenia and mycobacterial infection (MonoMAC) syndrome. Blood 2011, 118(10):2653-2655.
16. Ross E M, Moate P J, Marett L C, Cocks B G, Hayes B J: Metagenomic predictions: from microbiome to complex health and environmental phenotypes in humans and cattle. PloS one 2013, 8(9):e73056.
17. Endelman J B: Ridge Regression and Other Kernels for Genomic Selection with R Package rrBLUP. The Plant Genome 2011, 4(3):250-255.
18. Alcais A, Quintana-Murci L, Thaler D S, Schurr E, Abel L, Casanova J L: Life-threatening infectious diseases of childhood: single-gene inborn errors of immunity? Annals of the New York Academy of Sciences 2010, 1214:18-33.
19. Carneiro-Sampaio M, Coutinho A: Early-onset autoimmune disease as a manifestation of primary immunodeficiency. Frontiers in immunology 2015, 6:185.
20. Kemper K E, Reich C M, Bowman P J, Vander Jagt C J, Chamberlain A J, Mason B A, Hayes B J, Goddard M E: Improved precision of QTL mapping using a nonlinear Bayesian method in a multi-breed population leads to greater accuracy of across-breed genomic predictions. Genetics, selection, evolution: GSE 2015, 47:29.
21. MacLeod I M, Bowman P J, Vander Jagt C J, Haile-Mariam M, Kemper K E, Chamberlain A J, Schrooten C, Hayes B J, Goddard M E: Exploiting biological priors and sequence variants enhances QTL discovery and genomic prediction of complex traits. BMC genomics 2016, 17:144.
22. Kourou K, Exarchos T P, Exarchos K P, Karamouzis M V, Fotiadis D I Machine learning applications in cancer prognosis and prediction. Comput Struct Biotechnol J. 2014 Nov. 15; 13:8-17.
23. Han H, Liu Y. Transcriptome marker diagnostics using big data. IET Syst Biol. 2016 Feb.; 10(1):41-8.

1. A method for determining whether a subject has or is susceptible to developing a primary immunodeficiency (PID), the method comprising using a linear mixed model to fit a transcriptome profile of the subject to a PID prediction equation developed by fitting into a linear mixed model a transcriptomic relationship matrix generated from a reference set of transcriptome profiles of reference subjects with and without PID, wherein the prediction equation's result indicates whether the subject has or is susceptible to PID.

2. A method for developing a primary immunodeficiency (PID) prediction equation for determining whether a subject has or is susceptible to developing a PID, the method comprising fitting into a linear mixed model a transcriptomic relationship matrix generated from a reference set of transcriptome profiles of reference subjects with and without PID to develop the PID prediction equation.

3. The method of claim 1 or 2, further comprises measuring the transcriptome profile of the subject.

4. The method of any one of claims 1 to 3, further comprising measuring the transcriptome profiles of the reference subjects.

5. The method of any one of claims 1 to 4, wherein the linear mixed model is best linear unbiased prediction (BLUP), BayesR, or machine learning approaches.

6. The method of any one of claims 1 to 5, wherein the reference set further comprises a RNA sequence mutation profile.

7. The method of any one of claims 1 to 6, further comprising measuring a RNA sequence mutation profile of the subject for whom the determination of PID or susceptibility to PID is to be made.

8. The method of any one of claims 1 to 7, wherein the reference set further comprises a RNA sequence mutation profile and the linear mixed model is used to fit the transcriptome profile and a RNA sequence mutation profile of the subject to the PID prediction equation.

9. The method of any one of claims 1 to 8, wherein the reference set further comprises a DNA sequence mutation profile.

10. The method of any one of claims 1 to 9, further comprises measuring or determining the DNA sequence mutation profile of the subject for whom the determination of PID or susceptibility to PID is to be made

11. The method of any one of claims 1 to 10, wherein the reference set further comprises a DNA sequence mutation profile and the linear mixed model is used to fit the transcriptome profile and a DNA sequence mutation profile of the subject to the PID prediction equation.

12. The method of any one of claims 6 to 11, wherein the mutation profile comprises:
a) a RNA sequence of a PID gene comprising a known mutation resulting in a PID;
b) a new mutation, optionally a frameshift mutation, stop codon or amino acid change, that affects structure or function of a protein encoded by a known gene mutation of which results in a PID;
c) a dominant mutation in one allele that results in a PID;
d) two different mutations in the same gene, but on two different alleles that result in a PID;
e) a known mutation in RNA that is inferred or imputed by linkage to a co-occurring marker for a mutation resulting in a PID;
f) absence of expression of a gene normally expressed in non-PID subjects indicating a regulatory defect or destabilising mutation;
g) a defective exon structure indicating a splicing defect;
h) one or more, optionally one to three, additional mutations resulting in a PID; or
i) a sequence of more than one other gene, or an imputed sequence of more than one other gene, that contributes to PID severity.

13. The method of any one of claims 1 to 12, wherein the reference set further comprises a metagenome profile.

14. The method of any one of claims 1 to 13, further comprises measuring or determining the metagenome profile of the subject for whom the determination of PID or susceptibility to PID is to be made.

15. The method of any one of claims 1 to 13, wherein the reference set further comprises a metagenome profile and the linear mixed model is used to fit the transcriptome profile and a metagenome profile of the subject to the PID prediction equation.

16. The method of any one of claims 1 to 15, wherein the transcriptome profile or sequence mutation profile is obtained from sputum, blood, amniotic fluid, plasma, semen, bone marrow, tissue, urine, peritoneal fluid, or pleural fluid, optionally obtained by fine needle biopsy.

17. The method of claim 16, wherein the blood comprises peripheral blood mononuclear cells.

18. The method of claim 13 or claim 14, wherein the metagenome profile is obtained from a mouth swab, nose swab, throat swab, saliva, faeces, or skin.

19. The method of any one of claims 1 to 18, wherein the subject is human.

20. The method of any one of claims 1 to 19, wherein the profile of the subject for whom the determination of PID or susceptibility to PID is to be made is determined or measure from analysing a biological sample previously obtained from the subject.

21. A computer-implemented method for processing genomic information, the genomic information comprising a subject transcriptome profile, the method comprising:
accessing a reference set of transcriptome profiles of reference subjects, each reference subject either having or not having a primary immunodeficiency (PID);
generating a transcriptomic relationship matrix from the reference set of transcriptome profiles;
fitting the transcriptomic relationship matrix into a linear mixed model to generate a PID prediction equation; and
fitting the subject transcriptome profile to the PID prediction equation.

22. A computer-implemented method for generating a primary immunodeficiency (PID) prediction equation, the method comprising:
accessing a reference set of transcriptome profiles of reference subjects, each reference subject either having or not having a primary immunodeficiency (PID);
generating a transcriptomic relationship matrix from the reference set of transcriptome profiles; and
fitting the transcriptomic relationship matrix into a linear mixed model to generate the PID prediction equation.

23. The computer-implemented method of claim 21 or 22, further comprises measuring the transcriptome profile of the subject.

24. The computer-implemented method of any one of claims 21 to 23, further comprising measuring the transcriptome profiles of the reference subjects.

25. The computer-implemented method of any one of claims 21 to 24, wherein the linear mixed model is best linear unbiased prediction (BLUP), BayesR, random forest or machine learning approaches.

26. The computer-implemented method of any one of claims 21 to 25, wherein the reference set further comprises a RNA sequence mutation profile.

27. The computer-implemented method of claim 21, wherein the reference set further comprises a RNA sequence mutation profile and the linear mixed model is used to fit the transcriptome profile and a RNA sequence mutation profile of the subject to the PID prediction equation.

28. The computer-implemented method of any one of claims 21 to 25, wherein the reference set further comprises a DNA sequence mutation profile.

29. The computer-implemented method of any one of claims 21 to 28, wherein the reference set further comprises a DNA sequence mutation profile and the linear mixed model is used to fit the transcriptome profile and a DNA sequence mutation profile of the subject to the PID prediction equation.

30. The computer-implemented method of any one of claims 21 to 29, wherein the reference set further comprises a metagenome profile.

31. The computer-implemented method of claims 21 to 29, wherein the reference set further comprises a metagenome profile and the linear mixed model is used to fit the transcriptome profile and a metagenome profile of the subject to the PID prediction equation.

32. A non-transitory computer-readable medium storing instructions, which when executed by a processor cause the processor to:
access a reference set of transcriptome profiles of reference subjects, each reference subject either having or not having a primary immunodeficiency (PID);
generate a transcriptomic relationship matrix from the reference set of transcriptome profiles;
fit the transcriptomic relationship matrix into a linear mixed model to generate a PID prediction equation;
receive a subject transcriptome profile; and
fit the subject transcriptome profile to the PID prediction equation.

33. A non-transitory computer-readable medium storing instructions, which when executed by a processor cause the processor to:
access a reference set of transcriptome profiles of reference subjects, each reference subject either having or not having a primary immunodeficiency (PID);
generate a transcriptomic relationship matrix from the reference set of transcriptome profiles; and
fit the transcriptomic relationship matrix into a linear mixed model to generate the PID prediction equation.

34. The non-transitory computer-readable medium storing instructions of claims 32 to 33, wherein the linear mixed model is best linear unbiased prediction (BLUP), BayesR, random forest or machine learning approaches.

35. The non-transitory computer-readable medium storing instructions of any one of claims 32 to 34, wherein the reference set further comprises a RNA sequence mutation profile.

36. The non-transitory computer-readable medium storing instructions of claim 32, wherein the reference set further comprises a RNA sequence mutation profile and the linear mixed model is used to fit the transcriptome profile and a RNA sequence mutation profile of the subject to the PID prediction equation.

37. The non-transitory computer-readable medium storing instructions of any one of claims 32 to 36, wherein the reference set further comprises a DNA sequence mutation profile.

38. The non-transitory computer-readable medium storing instructions of any one of claims 32 to 36, wherein the reference set further comprises a DNA sequence mutation profile and the linear mixed model is used to fit the transcriptome profile and a DNA sequence mutation profile of the subject to the PID prediction equation.

39. The non-transitory computer-readable medium storing instructions of any one of claims 32 to 38, wherein the reference set further comprises a metagenome profile.

40. The non-transitory computer-readable medium storing instructions of claims 32 to 39, wherein the reference set further comprises a metagenome profile and the linear mixed model is used to fit the transcriptome profile and a metagenome profile of the subject to the PID prediction equation.
